(19) United States
(12) Patent Application Publication
(10) Pub. No.: US 2018/0265890 A1
(43) Pub. Date: Sep. 20, 2018

(54) EFFICIENT AND SAFE TRANSPOSON INTEGRATION SYSTEM AND USE THEREOF

(71) Applicants: SHANGHAI CELL THERAPY RESEARCH INSTITUTE, Shanghai (CN); SHANGHAI ENGINEERING RESEARCH CENTER FOR CELL THERAPY GROUP CO., LTD, Shanghai (CN)

(72) Inventors: Qijun QIAN, Shanghai (CN); Huajun JIN, Shanghai (CN); Linfang LI, Shanghai (CN); Tao LIU, Shanghai (CN); Minghui ZUO, Shanghai (CN); Hongping WU, Shanghai (CN); Mengchao WU, Shanghai (CN)

(73) Assignees: SHANGHAI CELL THERAPY RESEARCH INSTITUTE, Shanghai (CN); SHANGHAI ENGINEERING RESEARCH CENTER FOR CELL THERAPY GROUP CO., LTD, Shanghai (CN)

(21) Appl. No.: 15/764,840

(22) PCT Filed: Sep. 14, 2016
(86) PCT No.: PCT/CN2016/099005

§ 371 Date: Mar. 29, 2018

(30) Foreign Application Priority Data
Sep. 30, 2015 (CN) 201510638974.7

(51) Int. Cl. C12N 15/85; C12N 5/16; C12N 15/52; C12N 15/113

(52) U.S. Cl. CPC C12N 15/85; C12N 5/16; C12N 2800/107; C12N 15/113; C12N 2015/8518; C12N 15/52

(57) ABSTRACT

The invention belongs to the field of molecular biology, and relates to an efficient and safe transposon integration system and use thereof. The invention also relates to a nucleic acid construct and use thereof. Preferably, the nucleic acid construct comprises the following elements in order: a 5′-terminal repeat sequence of a transposon, a multiple cloning site, a polyA tailing signal sequence, a 3′-terminal repeat sequence of a transposon, a sequence encoding a transposase and a promoter controlling expression of the transposase; wherein the multiple cloning site is used for operably inserting an exogenous gene and optionally a promoter controlling expression of the exogenous gene; the polyA tailing signal sequence has a polyA tailing signal function in both forward and reverse directions; and the direction of the expression cassette of the transposase is opposite to the direction of the exogenous gene expression cassette. The nucleic acid construct is useful for mediating efficient and safe expression of an exogenous gene in a host cell.

### TECHNICAL FIELD

The invention belongs to the field of molecular biology, and relates to an efficient and safe transposon integration system and use thereof. The invention further relates to a nucleic acid construct and use thereof. The nucleic acid construct is useful for mediating efficient integration of, and efficient and stable expression of an exogenous gene in a host cell, wherein the integration sites are mainly present in 3 intergenic spacer regions in a host cell genome, which can avoid the risk resulting from random insertion to a large extent. The invention further relates to a recombinant vector and a recombinant host cell comprising the nucleic acid construct.

### BACKGROUND ART

Expression of an exogenous gene in a host cell can be classified into transient expression and stable expression, wherein stable expression refers to: (1) a eukaryotic cell is transfected with an exogenous gene and the exogenous gene is expressed after its integration into genome. The stable expression level of a recombinant gene is generally 1˜2 orders of magnitude lower than the transient expression level. (2) Although host cells are subjected to multiple passages or changed conditions, the expression level still remains stable.

Since stable expression can retain persistent expression of an exogenous gene for a long time with cell division, which is of important significance in ex vivo cell modification, such as research on Chimeric Antigen Receptor T-Cell Immunotherapy (CAR-T). CAR-T cells can specifically recognize and efficiently kill tumor cells expressing specific cell surface antigen, and have achieved significant therapeutic effect in clinic. For example, CAR-T cells against CD19 can efficiently kill B cell lymphoma expressing CD19 surface antigen, and have an effective remission rate of up to 90% for patients with advanced-stage refractory B cell lymphoma (Maude SL, Frey N, Shaw PA, Aplenc R, Barrett D M, Bunin N J, Chew A, Gonzalez V E, Zheng Z, Lacey S F, Mahnke Y D, Melenhorst J J, Rheingold S R, Shen A, Teachey D T, Levine B L, June C H, Porter DL, Grupp S A. Chimeric antigen receptor T cells for sustained remissions in leukemia. N Engl J Med. 2014; 371(16): 1507-17).

In order to achieve stable expression of an exogenous gene in a host cell, the commonly used vector systems include: 1. Retrovirus system: it can effectively transfect a host cell, and mediate efficient integration of an exogenous gene expression cassette into a genome, however, it has a limited loading capacity, and the process for preparing retrovirus particles is complex. 2. Eukaryotic expression plasmid system: its preparation process is relatively simple, but it inserts an exogenous gene into a host genome by means of random DNA recombination, and therefore has a very low integration rate. 3. Transposon system: it uses a plasmid system, its preparation process is relatively simple, and it has an exogenous gene integrated into a genome via transposase, with a relatively low integration rate.

The earliest transposon system applied to mammal is “Sleeping Beauty” transposon derived from fish, however, due to the defects such as overexpression inhibition effect and the small size of the carried fragment (about 5 kb), the application of “Sleeping Beauty” transposon is restricted in transgenic application. The piggyBac (PB) transposon derived from lepidoptera insect is the most active transposon in mammal now. It can work in a very wide range of hosts, including from unicellular organism to mammal; and can carry a relatively large exogenous DNA fragment, with a transposition efficiency not decreased significantly when the transposed fragment is no more than 14 kb in size. PB transposon achieves transposition mainly via a “cut-paste” mechanism, wherein after the transposed fragment is cut off, no footprint will be left at the original site, and the genome can be repaired precisely after the cut; and PB transposon plays an important role in application of reversible transgene. In addition, PB transposase is highly flexible, and it cannot only change the activity and the action mode of transposase, but also enhance the targeting potency of the transposition of an exogenous gene, by the fusion with an additional functional protein or by changing the functional regions of transposase. In recent years, the integration efficiency of PB is further increased in mammalian cells by codon optimization, site-specific mutagenesis of amino acids at specific sites, introduction of a corresponding nuclear localization tag, and the like, and thereby the system is widely applied in the fields such as genome research, gene therapy, cell therapy, stem cell induction and post-induction differentiation.

A traditional PB transposon system uses a binary transposition system consisting of a donor plasmid (terminal repeat sequences recognized by PB integrase may locate at both ends of an exogenous gene expression cassette) and a transposase helper plasmid (which provides PB transposase). In the binary transposition system, in order to achieve the effective integration of an exogenous gene expression cassette, the two plasmids must be transfected into the same cell, however, during transfection, only a portion of cells are co-transfected with the two plasmids (the other cells are either transfected with none of the plasmids, or are transfected with only one of the plasmids, neither of which can achieve effective integration), which reduces the integration efficiency to some extent. In addition, since PB transposon system works in a completely reversible “cut-paste” manner, it is still possible to cut off the exogenous gene expression cassette that has been integrated into the genome as long as the integrase is expressed persistently, which renders the genome unstable, and actually reduces the integration efficiency.

Therefore, in order to improve the integration efficiency of PB transposon system, it is very necessary to engineer the vector system, by bringing a donor plasmid and a transposase helper plasmid into the same plasmid, and meanwhile providing a mechanism of self-inactivating transposase to ensure that the expression of transposase can be terminated just in time after the integration of an exogenous gene is accomplished. It is reported in documents that one strategy is to place a promoter controlling PB expression between an exogenous expression cassette and one of transposase ITRs, once the exogenous gene expression cassette is cut off from the plasmid and is integrated into genome, transcription will be stopped for lack of a promoter in PB expression cassette, and the expression is terminated in time (Urschitz J, Kawasumi M, Owens J, Morozumi K, Yamashiro H, Stoytchev L Marh J, Dee J A, Kawamoto K, Coates C J, Kaminski J M, Pelczar P, Yanagimachi R, Moisyadi S. Helper-independent piggyBac plasmids for gene delivery approaches: strategies for avoiding potential genotoxic effects. Proc Natl Acad Sci U S A. 2010; 107(18): 8117-22.). However, such a strategy has the defect that the integration of a strong promoter controlling the expression of PB gene in genome might activate the expression of the flanking gene of the integration site in a host cell, and therefore has a potential risk in safety.

Another strategy is that PB expression cassette and an exogenous gene expression cassette are placed in the same direction, and share the same PolyA tailing signal sequence, once the exogenous gene expression cassette is cut off from the plasmid and is integrated into genome, due to lack of PolyA tailing signal sequence in PB expression cassette, the transcribed mRNA will be unstable and degrades rapidly, and the expression of PB is terminated (Chakraborty S, Ji H, Chen J, Gersbach C A, Leong K W. Vector modifications to eliminate transposase expression following piggyBac-mediated transgenesis. Sci Rep. 2014; 4: 7403). Such a strategy has the defect that the mRNA product transcribed by the PB gene expression cassette actually covers the whole exogenous gene expression cassette sequence, and if the exogenous gene expression cassette is large, its mRNA will also be large in length, which results in a decreased PB transcription efficiency, and makes it difficult to reach a PB expression level necessary for integration.

In a unary transposition system, an exogenous gene expression cassette and PB expression cassette must be packaged into the same vector, however, since the sequence encoding PB is long, about 2 kb in length, it results in large plasmid fragments and greatly reduces the transfection efficiency.

In addition, the integration sites of currently available PB transposon systems tend to locate in coding genes (Woodard L E, Wilson M H. Trends Biotechnol. piggyBac-ing models and new therapeutic strategies. 2015; 33(9): 525-33. See Table 1 on page 4). The coding gene described here is a concept relative to a non-coding gene, which refers to a gene capable of encoding a protein having a corresponding function; if a tumor-associated gene is insertionally inactivated, or activated abnormally, it may result in a risk of carcinogenesis.

### CONTENTS OF INVENTION

Through a large amount of experimentations and creative work, the inventors constructed a PiggyBac transposon-based integration system, which can mediate efficient integration of, and efficient and stable expression of an exogenous gene in a host cell. The inventor surprisingly found that the sites of exogenous gene integration mediated by the system mainly locate in 3 intergenic spacer regions in a host cell genome, which can avoid the risk resulting from random insertion to a large extent. Thereby, the following invention is provided.

In an aspect, the invention relates to a nucleic acid construct, comprising the following elements in order:

a 5′-terminal repeat sequence of a transposon, a polyA tailing signal sequence, a 3′-terminal repeat sequence of a transposon, a sequence encoding a transposase and a promoter controlling expression of the transposase;

wherein, the polyA tailing signal sequence has a polyA tailing signal function in both forward and reverse directions;

the direction of the expression cassette of the transposase is opposite to the direction of the exogenous gene expression cassette.

In an aspect, the invention relates to a nucleic acid construct, comprising the following 6 elements in order:

a 5′-terminal repeat sequence of a transposon, a multiple cloning site, a polyA tailing signal sequence, a 3′-terminal repeat sequence of a transposon, a sequence encoding a transposase and a promoter controlling expression of the transposase;

wherein,

the multiple cloning site is used for operably inserting an exogenous gene and optionally a promoter controlling expression of the exogenous gene;

the polyA tailing signal sequence has a polyA tailing signal function in both forward and reverse directions;

the direction of the expression cassette of he transposase is opposite to the direction of the exogenous gene expression cassette.

In the invention, if not specially indicated, the direction of an exogenous gene expression cassette is taken as a forward direction, and the direction of a transposase expression cassette is taken as a reverse direction.

In the expression “comprising the following elements in order”, “in order” refers to a direction and/or order from upstream to downstream. In the invention, if not specially indicated, along the “forward” direction refers to from upstream to downstream, and along the “reverse” direction refers to from downstream to upstream.

In an embodiment of the invention, the 6 elements are independently single-copy or multi-copy.

The 6 elements can be connected to each other directly, or an additional sequence such as a linker or an enzyme cleavage site may also be comprised.

In an embodiment of the invention, the nucleic acid construct, wherein the polyA tailing signal sequence is a polyA tailing signal sequence that has a polyA tailing signal function in both forward and reverse directions; or consists of two polyA tailing signal sequences which are connected to each other in an opposite direction and each has a monodirectional polyA tailing signal.

In the invention, if not specially indicated, the expression “the polyA tailing signal sequence has a polyA tailing signal function in both forward and reverse directions” includes, but is not limited to the following circumstances:

1) a polyA tailing signal sequence, which has a polyA tailing signal function in both forward and reverse directions; and

2) two polyA tailing signal sequences, wherein one has a polyA tailing signal function in a forward direction, and the other has a polyA tailing signal function in a reverse direction.

Preferably, the technical solution in Item 1) is used. Without restriction by theory, such an exogenous gene expression cassette and PiggyBac transposase expression cassette can share a polyA tailing signal sequence, thereby saving one polyA tailing signal sequence, which reflects the principle of intensivism, reduces the size of plasmids, and under the precondition of ensuring transfection efficiency, is favorable for increasing the capacity of an exogenous gene expression cassette.

In another non-preferred technical solution of the invention, PB expression cassette and an exogenous gene expression cassette are placed in the same direction, and two polyA tailing signal sequences are used, wherein the PB expression cassette is placed upstream, and its polyA tailing signal sequence is placed between an ITR and an exogenous gene promoter. For example: a promoter controlling the expression of PB transposase, a sequence encoding a PB transposase, a 5′-terminal repeat sequence of a transposon, a polyA tailing signal sequence 1, an exogenous gene promoter and an exogenous gene (multiple cloning site), a polyA tailing signal sequence 2, and a 3′-terminal repeat sequence of a transposon are arranged in this order; and the direction of the PB transposase expression cassette is the same as the direction of the exogenous gene expression cassette.

The nucleic acid construct according to the invention, wherein, the transposon is one or more selected from the group consisting of PiggyBac, sleeping beauty, frog prince, Tn5 and Ty; and preferably, the transposon is a PiggyBac transposon.

The nucleic acid construct according to the invention, wherein, the position of the 5′-terminal repeat sequence of a transposon is interchangeable with the position of the 3′ -terminal repeat sequence of a transposon.

The nucleic acid construct according to the invention, wherein,

the 5′-terminal repeat sequence of a transposon is 5′-terminal repeat sequence of a transposon PiggyBac; the 3′-terminal repeat sequence of a transposon is 3′-terminal repeat sequence of a PiggyBac transposon; the transposase is a PiggyBac transposase.

The nucleic acid construct according to the invention, wherein

the 5′-terminal repeat sequence of a PiggyBac transposon has a nucleotide sequence set forth in SEQ ID NO: 1; and/or the 3′-terminal repeat sequence of a PiggyBac transposon has a nucleotide sequence set forth in SEQ ID NO: 4.

The nucleic acid construct according to the invention, wherein,

the amino acid sequence of the PiggyBac transposase has an amino acid sequence set forth in SEQ ID NO: 17; preferably, the PiggyBac transposase is encoded by a nucleotide sequence set forth in SEQ ID NO: 5.

The nucleic acid construct according to the invention, wherein,

the sequence encoding a transposase comprises or is operably linked to a single copy of or multiple copies of a sequence encoding a nuclear localization signal; preferably, the sequence encoding a nuclear localization signal is a sequence encoding a c-myc nuclear localization signal, for example, a sequence set forth in SEQ ID NO: 18. Nuclear localization signal can induce aggregation of transposase in nuclei, thereby improving transposition efficiency.

The nucleic acid construct according to the invention, is characterized by one or more of the following items (1)-(3):

(1) the multiple cloning site has a nucleotide sequence set forth in SEQ ID NO: 2;

(2) the polyA tailing signal sequence has a nucleotide sequence set forth in SEQ ID NO: 3;

the sequence set forth in SEQ ID NO: 3 has a polyA tailing signal function in both forward and reverse directions.

(3) the promoter is selected from the group consisting of CMV promoter (for example, as set forth in SEQ ID NO: 6), EF1α promoter, SV40 promoter, Ubiquitin B promoter, CAG promoter, HSP70 promoter, PGK-1 promoter, β-actin promoter, TK promoter and GRP78 promoter.

The nucleic acid construct according to the invention, wherein the nucleic acid construct is operably linked to one or more identical or different exogenous genes and optionally a promoter controlling expression of the one or more exogenous genes, or operably has one or more identical or different exogenous genes and optionally a promoter controlling expression of the exogenous gene inserted (for example, at a multiple cloning site); or wherein the multiple cloning site is replaced by one or more identical or different exogenous genes and optionally a promoter controlling expression of the one or more exogenous genes; and each exogenous gene is independently of single-copy or multi-copy;

preferably, the exogenous gene is one or more selected from the group consisting of a gene encoding a fluorescein reporter (such as green fluorescent protein, red fluorescent protein, and yellow fluorescent protein, etc.), a gene encoding luciferase (such as firefly luciferase, and ranilla luciferase), a gene encoding a naturally occurring functional protein (for example, TP53, GM-CST, OCT4, SOX2, Nanog, KLF4, c-Myc), RNAi gene and artificial chimeric gene (for example, chimeric antigen receptor gene such as CAR19, Fc fusion protein gene, and full-length antibody gene);

preferably, the exogenous gene has a sequence set forth in any one or more of SEQ ID NO: 9-11 and 16.

In a further aspect, the invention relates to a recombinant vector, comprising the nucleic acid construct according to the invention;

preferably, the recombinant vector is a recombinant cloning vector, a recombinant eukaryotic expression vector or a recombinant virus vector;

preferably, the recombinant cloning vector is a recombinant vector obtained by recombination of the nucleic acid construct according to the invention with pUC18, pUC19, pMD18-T, pMD19-T, pGM-T vector, pUC57, pMAX or pDC315 serial vector;

preferably, the recombinant eukaryotic expression vector is a recombinant vector obtained by recombination of the nucleic acid construct according to the invention with pCDNA3 serial vector, pCDNA4 serial vector, pCDNA5 serial vector, pCDNA6 serial vector, pRL serial vector, pUC57 vector, pMAX vector or pDC315 serial vector;

preferably, the recombinant virus vector is a recombinant adenovirus vector, a recombinant adeno-associated virus vector, a recombinant retrovirus vector, a recombinant herpes simplex virus vector or a recombinant vaccinia virus vector.

In a further aspect, the invention relates to a recombinant host cell, comprising the nucleic acid construct according to the invention or the recombinant vector according to the invention; preferably, the recombinant host cell is a recombinant mammalian cell; for example, a recombinant primary T cell, a Jurkat cell, K562 cell, a stem cell, a tumor cell, a HEK293 cell or a CHO cell; preferably, the stem cell is an embryonic stem cell.

The nucleic acid construct, recombinant vector or recombinant host cell according to the invention, for use in integration of an exogenous gene expression cassette into a host cell genome.

In a further aspect, the invention relates to use of the nucleic acid construct according to the invention, the recombinant vector according to the invention or the recombinant host cell according to the invention, which is selected from any one of the following (1)-(4):

(1) use for manufacture of, or as, a medicament or agent for the integration of an exogenous gene expression cassette into a host cell genome; preferably, the host cell is a mammalian cell, for example, a primary T cell, a Jurkat cell, a K562 cell, a stem cell, a tumor cell, a HEK293 cell or a CHO cell; preferably, the stem cell is an embryonic stem cell;

(2) use for manufacture of, or as, a tool for the integration of an exogenous gene expression cassette into a host cell genome; preferably, the host cell is a mammalian cell, for example, a primary T cell, a Jurkat cell, a K562 cell, a stem cell, a tumor cell, a HEK293 cell or a CHO cell; preferably, the stem cell is an embryonic stem cell;

(3) use for manufacture of, or as, a medicament or a formulation for genome research, gene therapy, cell therapy or stem cell induction and post-induction differentiation; preferably, the stem cell is an embryonic stem cell;

(4) use for manufacture of, or as, a tool for genome research, gene therapy, cell therapy or stem cell induction and post-induction differentiation; preferably, the stem cell is an embryonic stem cell.

Said use can be achieved by insertion of an exogenous gene having the corresponding function, the exogenous gene having the corresponding function has a function corresponding to a particular use, for example, a therapeutic function or an inducing function.

In a further aspect, the invention relates to a method for introducing the nucleic acid construct or the recombinant vector according to the invention into a mammalian cell, including virus-mediated transformation, microinjection, particle bombardment, gene gun transformation, electroporation, etc. In an embodiment of the invention, the method is electroporation.

In a further aspect, the invention relates to a method for integrating an exogenous gene expression cassette into a host cell genome, comprising the step of integrating an exogenous gene expression cassette into a host cell genome by using the nucleic acid construct, recombinant vector or recombinant host cell according to the invention. In an embodiment of the invention, in the method, the nucleic acid construct according to the invention or the recombinant vector according to the invention is introduced into a host cell such as a mammalian cell by a method selected from the group consisting of virus-mediated transformation, microinjection, particle bombardment, gene gun transformation and electroporation.

The terms involved in the invention are explained as follows.

In the invention, the term “expression cassette” refers to a complete set of elements necessary for the expression of a gene, including a promoter, a gene, and a PolyA tailing signal sequence.

The term “nucleic acid construct” used herein refers to a single-stranded or double-stranded nucleic acid molecule, preferably an artificially constructed nucleic acid molecule. Optionally, the nucleic acid construct further comprises one or more operably linked regulatory sequences, which can direct the expression of a coding sequence in a suitable host cell under compatible conditions. Expression should be construed as any steps involved in the production of a protein or a polypeptide, including, but not limited to transcription, post-transcriptional modification, translation, post-translational modification, and secretion.

The term “operably inserting/linking” used herein refers to such a configuration in which a regulatory sequence is located at a suitable position relative to the DNA sequence, so that the regulatory sequence can direct the expression of a protein or polypeptide. In the nucleic acid construct of the invention, for example, an exogenous gene promoter and an exogenous gene are placed in the multiple cloning site by DNA recombination technique. The “operably linking” can be achieved by means of DNA recombination, preferably, the nucleic acid construct is a recombinant nucleic acid construct.

The term “coding sequence” used herein refers to the part of a nucleic acid sequence that directly determines the amino acid sequence of its protein product. The boundaries of a coding sequence are generally defined by a ribosome binding site (for prokaryotic cells) adjacent to upstream of mRNA 5′-terminal open reading frame and a transcription termination sequence adjacent to downstream of mRNA 3′-terminal open reading frame. Coding sequence includes, but is not limited to DNA, cDNA and a recombinant nucleic acid sequence.

The term “regulatory sequence” used herein refers to all the components necessary or favorable for the expression of the peptide of the invention. Each regulatory sequence may be naturally endogenous or exogenous to the nucleic acid sequence encoding protein or polypeptide. These regulatory sequences include, but are not limited to leader sequence, polyadenylation sequence, propeptide-encoding sequence, promoter, signal sequence, and transcription terminator. A regulatory sequence comprises at least a promoter as well as a transcription and translation termination signal. A regulatory sequence carrying a linker can be provided to introduce a specific restriction site so that the regulatory sequence can be linked to the coding region of the nucleic acid sequence encoding protein or polypeptide.

A regulatory sequence can be a suitable promoter sequence, i.e. a nucleic acid sequence that can be recognized by a host cell expressing the nucleic acid sequence. A promoter sequence comprises transcription regulating sequence(s) mediating the expression of protein or polypeptide. A promoter can be any nucleic acid sequence having transcription activity in the selected host cell, including mutated, truncated, and hybrid promoter, and can be obtained from a gene encoding extracellular or endocellular protein or polypeptide endogenous or exogenous to the host cell.

A regulatory sequence can also be a suitable transcription termination sequence, i.e. a sequence that can be recognized by a host cell and thereby the transcription is terminated. A termination sequence is operably linked to 3′ end of the nucleic acid sequence encoding protein or polypeptide. Any terminator can be used in the invention as long as it can function in the selected host cell.

A regulatory sequence can also be a suitable leader sequence, i.e. untranslated region of mRNA that plays a very important role in translation in a host cell. A leader sequence is operably linked to 5′ end of the nucleic acid sequence encoding polypeptide. Any leader sequence can be used in the invention as long as it can function in the selected host cell.

A regulatory sequence can also be a signal peptide coding region, which encodes an amino acid sequence linked to the N-terminal of a protein or a polypeptide, and can guide coding polypeptide into cell secretory pathway. The 5′ end of the coding region in a nucleic acid sequence might naturally comprise a signal peptide coding region that is consistent with the reading frame for translation and naturally linked to the coding region of the secretory polypeptide. Alternatively, 5′ end of a coding region may comprise a signal peptide coding region that is exogenous to the coding sequence. When the coding sequence does not comprise a signal peptide coding region under normal conditions, an exogenous signal peptide coding region might need to be added. Alternatively, a natural signal peptide coding region may be simply replaced with an exogenous signal peptide coding region in order to enhance polypeptide secretion. However, any signal peptide coding region can be used in the invention as long as it can guide the expressed polypeptide into the secretory pathway of the used host cell.

A regulatory sequence may also be a propeptide coding region, which encodes an amino acid sequence located at N terminal of a polypeptide. The polypeptide obtained is called proenzyme or propolypeptide. A propolypeptide is generally inactive, but can be converted to a mature active polypeptide by the excision of a propeptide from a propolypeptide via catalysis or self-catalysis.

When a signal peptide and a propeptide region are both present in the N-terminal of a polypeptide, the propeptide region is closely adjacent to the N-terminal of the polypeptide, while the signal peptide region is closely adjacent to the N-terminal of the propeptide region.

It may also be necessary to add a regulatory sequence that can regulate the expression of a polypeptide depending on the growth of a host cell. Examples of regulatory system are systems that can respond to chemical or physical stimuli (including where a regulatory compound is present), so as to initiate or terminate gene expression. Other examples of regulatory sequence are the regulatory sequences that enable the amplification of gene. In these examples, the nucleic acid sequence encoding a protein or polypeptide should be operably linked to a regulatory sequence.

### Beneficial Effects of the Invention

The invention has one or more of the following technical effects:

(1) the invention provides a PiggyBac transposon-based efficient integration system, which can mediate efficient integration of an exogenous gene into a host cell genome, and enable stable expression; and

(2) the inventors surprisingly found that the insertion sites of the system into a host genome have an obvious preference, i.e. the sites of exogenous gene integration mediated thereby are mainly present in 3 intergenic spacer regions in a host cell genome, which can avoid the risk resulting from random insertion to a large extent.

### DESCRIPTION OF THE DRAWINGS

FIG. 1 shows pNB vector map.

FIG. 2 shows the relative expression level of PB gene-time curve after transfection of Jurkat cells with pNB.

FIG. 3 shows the EGFP positive cell percent-time curve after transfection of Jurkat cells with pN:328-EGFP.

FIG. 4 shows the fluorograms of 4 cells transfected with pNB328-EGFP. FIG. 4A-FIG. 4B refer to Jurkat cells, FIG. 4C-FIG. 4D refer to K562 cells, FIG. 4E-FIG. 4F refer to primary T cells, and FIG. 4G-FIG. 4H refer to mouse embryonic stem (ES) cells, wherein, the left FIG. 4A, FIG. 4C, FIG. 4E, and FIG. 4G were photos taken in white light, which showed cell morphology; and the right FIG. 4B, FIG. 4D, FIG. 4F, and FIG. 4H were photos taken in fluorescent light, which showed green fluorescence. For a same kind of cell, the left and right photos were taken in a same visual field.

FIG. 5 illustrates the flow cytometry of Jurkat cell (FIG. 5A), K562 cell (FIG. 5B), primary T cell (FIG. 5C), and mouse ES cell (FIG. 5D) transfected with pNB328-EGFP.

FIG. 6 illustrates luciferase assay of Huh7 cell transfected with pNB328-luc.

FIG. 7 illustrates fluorescence intensity assay on the expression of EGFP gene after transfection of primary T cell with pNB328-EGFP. FIGS. 7A and FIG. 7C were photos taken in white light, which showed cell morphology; and FIGS. 7B and FIG. 7D were photos taken in fluorescent light, which showed green fluorescence.

FIG. 8 illustrates the analysis of integration sites after the transfection of primary T cell with pNB328-EGFP. The circled portions are the integration hotspots. FIGS. 8A, FIG. 8B, and FIG. 8C represent the determination of integration sites of normal human primary T cells from three different sources, respectively. A triangle represents an integration site that is present in an intragenic region, an arrow represents an integration site that is present in an intragenic region, and a circle represents an integration hotspot.

FIG. 9 illustrates the killing effect on Raji cell after transfection of primary T cell with pNB328-CAR19.

### SEQUENCE INFORMATION

### Sequence 1 (SEQ ID NO: 1, 67bp), 5′-Terminal Repeat Sequence of a PiggyBac Transposon

TTAACCCTAGAAAGATAATCATATTGTGACGTACGTTAAAGATAATCATG
CGTAAAATTGACGCATG

### Sequence 2 (SEQ ID NO: 2, 51bp), Multiple Cloning Site

TCTAGAGTCGAATTCTGAGCTAGCGATGGATCCTGCACTAGTGCTGTCGA
C

### Sequence 3 (SEQ ID NO: 3, 222bp), PolyA Tailing Signal Sequence

CAGACATGATAAGATACATTGATGAGTTTGGACAAACCACAACTAGAATG
CAGTGAAAAAAATGCTTTATTTGTGAAATTTGTGATGCTATTGCTTTATT
TGTAACCATTATAAGCTGCAATAAACAAGTTAACAACAACAATTGCATTC
ATTTTATGTTTCAGGTTCAGGGGGAGGTGTGGGAGGTTTTTTAAAGCAAG
TAAAACCTCTACAAATGTGGTA

### Sequence 4 (SEQ ID NO: 4, 40bp), 3′-Terminal Repeat Sequence of a PiggyBac Transposon

GCATGCGTCAATTTTACGCAGACTATCTTTCTAGGGTTAA

### Sequence 5 (SEQ ID NO: 5, 1815bp), Sequence Encoding PiggyBac Transposase and Comprising a Sequence Encoding a C-Myc Nuclear Localization Signal, Wherein the Underlined Part is a Sequence Encoding a C-Myc Nuclear Localization Signal

ATGGGCCCTGCTGCCAAGAGGGTCAAGTTGGACGGCAGCAGCCTGGACGA
CGAGCACATCCTGAGCGCCCTGCTGCAGAGCGACGACGAGCTGGTGGGCG
AGGACAGCGACAGCGAGGTGAGCGACCACGTGAGCGAGGACGACGTGCAG
AGCGACACCGAGGAGGCCTTCATCGACGAGGTGCACGAGGTGCAGCCCAC
CAGCAGCGGCAGCGAGATCCTGGACGAGCAGAACGTGATCGAGCAGCCCG
GCAGCAGCCTGGCCAGCAACCGCATCCTGACCCTGCCCCAGCGCACCATC
CGCGGCAAGAACAAGCACTGCTGGAGCACCAGCAAGCCCACCCGCCGCAG
CCGCGTGAGCGCCCTGAACATCGTGCGCAGCCAGCGCGGCCCCACCCGCA
TGTGCCGCAACATCTACGACCCCCTGCTGTGCTTCAAGCTGTTCTTCACC
GACGAGATCATCAGCGAGATCGTGAAGTGGACCAACGCCGAGATCAGCCT
GAAGCGCCGCGAGAGCATGACCAGCGCCACCTTCCGCGACACCAACGAGG
ACGAGATCTACGCCTTCTTCGGCATCCTGGTGATGACCGCCGTGCGCAAG
GACAACCACATGAGCACCGACGACCTGTTCGACCGCAGCCTGAGCATGGT
GTACGTGAGCGTGATGAGCCGCGACCGCTTCGACTTCCTGATCCGCTGCC
TGCGCATGGACGACAAGAGCATCCGCCCCACCCTGCGCGAGAACGACGTG
TTCACCCCCGTGCGCAAGATCTGGGACCTGTTCATCCACCAGTGCATCCA
GAACTACACCCCCGGCGCCCACCTGACCATCGACGAGCAGCTGCTGGGCT
TCCGCGGCCGCTGCCCCTTCCGCGTGTACATCCCCAACAAGCCCAGCAAG
TACGGCATCAAGATCCTGATGATGTGCGACAGCGGCACCAAGTACATGAT
CAACGGCATGCCCTACCTGGGCCGCGGCACCCAGACCAACGGCGTGCCCC
TGGGCGAGTACTACGTGAAGGAGCTGAGCAAGCCCGTGCACGGCAGCTGC
CGCAACATCACCTGCGACAACTGGTTCACCAGCATCCCCCTGGCCAAGAA
CCTGCTGCAGGAGCCCTACAAGCTGACCATCGTGGGCACCGTGCGCAGCA
ACAAGCGCGAGATCCCCGAGGTGCTGAAGAACAGCCGCAGCCGCCCCGTG
GGCACCAGCATGTTCTGCTTCGACGGCCCCCTGACCCTGGTGAGCTACAA
GCCCAAGCCCGCCAAGATGGTGTACCTGCTGAGCAGCTGCGACGAGGACG
CCAGCATCAACGAGAGCACCGGCAAGCCCCAGATGGTGATGTACTACAAC
CAGACCAAGGGCGGCGTGGACACCCTGGACCAGATGTGCAGCGTGATGAC
CTGCAGCCGCAAGACCAACCGCTGGCCCATGGCCCTGCTGTACGGCATGA
TCAACATCGCCTGCATCAACAGCTTCATCATCTACAGCCACAACGTGAGC
AGCAAGGGCGAGAAGGTGCAGAGCCGCAAGAAGTTCATGCGCAACCTGTA
CATGGGCCTGACCAGCAGCTTCATGCGCAAGCGCCTGGAGGCCCCCACCC
TGAAGCGCTACCTGCGCGACAACATCAGCAACATCCTGCCCAAGGAGGTG
CCCGGCACCAGCGACGACAGCACCGAGGAGCCCGTGATGAAGAAGCGCAC
CTACTGCACCTACTGCCCCAGCAAGATCCGCCGCAAGGCCAGCGCCAGCT
GCAAGAAGTGCAAGAAGGTGATCTGCCGCGAGCACAACATCGACATGTGC
CAGAGCTGCTTCTAA

### Sequence 6 (SEQ ID NO: 6, 531bp), CMV Promoter

ATATACTGAGTCATTAGGGACTTTCCAATGGGTTTTGCCCAGTACATAAG
GTCAATAGGGGTGAATCAACAGGAAAGTCCCATTGGAGCCAAGTACACTG
AGTCAATAGGGACTTTCCATTGGGTTTTGCCCAGTACAAAAGGTCAATAG
GGGGTGAGTCAATGGGTTTTTCCCATTATTGGCACGTACATAAGGTCAAT
AGGGGTGAGTCATTGGGTTTTTCCAGCCATTAAATTAAAACGCCATGTAC
TTTCCCACCATTGACGTCAATGGGCTATTGAAACTAATGCAACGTGACCT
TTAAACGGTACTTTCCCATAGCTGATTAATGGGAAAGTACCGTTCTCGAG
CCAATACACGTCAATGGGAAGTGAAAGGGCAGCCAAAACGTAACACCGCC
CCGGTTTTCCCCTGGAAATTCCATATTGGCACTCATTCTATTGGCTGAGC
TGCGTTCTACGTGGGTATAAGAGGCGCGACCAGCGTCGGTACCGTCGCAG
TCTTCGGTCTGACCACCGTAGAACGCAGATC

### Sequence 7 (SEQ ID NO: 7, 2760bp), a long sequence connected in Example 1

GGCGCGCCTTAACCCTAGAAAGATAATCATATTGTGACGTACGTTAAAGA
TAATCATGCGTAAAATTGACGCATGTCTAGAGTCGAATTCTGAGCTAGCG
ATGGATCCTGCACTAGTGCTGTCGACCAGACATGATAAGATACATTGATG
AGTTTGGACAAACCACAACTAGAATGCAGTGAAAAAAATGCTTTATTTGT
GAAATTTGTGATGCTATTGCTTTATTTGTAACCATTATAAGCTGCAATAA
ACAAGTTAACAACAACAATTGCATTCATTTTATGTTTCAGGTTCAGGGGG
AGGTGTGGGAGGTTTTTTAAAGCAAGTAAAACCTCTACAAATGTGGTAGC
ATGCGTCAATTTTACGCAGACTATCTTTCTAGGGTTAAATCGATTTAGAA
GCAGCTCTGGCACATGTCGATGTTGTGCTCGCGGCAGATCACCTTCTTGC
ACTTCTTGCAGCTGGCGCTGGCCTTGCGGCGGATCTTGCTGGGGCAGTAG
GTGCAGTAGGTGCGCTTCTTCATCACGGGCTCCTCGGTGCTGTCGTCGCT
GGTGCCGGGCACCTCCTTGGGCAGGATGTTGCTGATGTTGTCGCGCAGGT
AGCGCTTCAGGGTGGGGGCCTCCAGGCGCTTGCGCATGAAGCTGCTGGTC
AGGCCCATGTACAGGTTGCGCATGAACTTCTTGCGGCTCTGCACCTTCTC
GCCCTTGCTGCTCACGTTGTGGCTGTAGATGATGAAGCTGTTGATGCAGG
CGATGTTGATCATGCCGTACAGCAGGGCCATGGGCCAGCGGTTGGTCTTG
CGGCTGCAGGTCATCACGCTGCACATCTGGTCCAGGGTGTCCACGCCGCC
CTTGGTCTGGTTGTAGTACATCACCATCTGGGGCTTGCCGGTGCTCTCGT
TGATGCTGGCGTCCTCGTCGCAGCTGCTCAGCAGGTACACCATCTTGGCG
GGCTTGGGCTTGTAGCTCACCAGGGTCAGGGGGCCGTCGAAGCAGAACAT
GCTGGTGCCCACGGGGCGGCTGCGGCTGTTCTTCAGCACCTCGGGGATCT
CGCGCTTGTTGCTGCGCACGGTGCCCACGATGGTCAGCTTGTAGGGCTCC
TGCAGCAGGTTCTTGGCCAGGGGGATGCTGGTGAACCAGTTGTCGCAGGT
GATGTTGCGGCAGCTGCCGTGCACGGGCTTGCTCAGCTCCTTCACGTAGT
ACTCGCCCAGGGGCACGCCGTTGGTCTGGGTGCCGCGGCCCAGGTAGGGC
ATGCCGTTGATCATGTACTTGGTGCCGCTGTCGCACATCATCAGGATCTT
GATGCCGTACTTGCTGGGCTTGTTGGGGATGTACACGCGGAAGGGGCAGC
GGCCGCGGAAGCCCAGCAGCTGCTCGTCGATGGTCAGGTGGGCGCCGGGG
GTGTAGTTCTGGATGCACTGGTGGATGAACAGGTCCCAGATCTTGCGCAC
GGGGGTGAACACGTCGTTCTCGCGCAGGGTGGGGCGGATGCTCTTGTCGT
CCATGCGCAGGCAGCGGATCAGGAAGTCGAAGCGGTCGCGGCTCATCACG
CTCACGTACACCATGCTCAGGCTGCGGTCGAACAGGTCGTCGGTGCTCAT
GTGGTTGTCCTTGCGCACGGCGGTCATCACCAGGATGCCGAAGAAGGCGT
AGATCTCGTCCTCGTTGGTGTCGCGGAAGGTGGCGCTGGTCATGCTCTCG
CGGCGCTTCAGGCTGATCTCGGCGTTGGTCCACTTCACGATCTCGCTGAT
GATCTCGTCGGTGAAGAACAGCTTGAAGCACAGCAGGGGGTCGTAGATGT
TGCGGCACATGCGGGTGGGGCCGCGCTGGCTGCGCACGATGTTCAGGGCG
CTCACGCGGCTGCGGCGGGTGGGCTTGCTGGTGCTCCAGCAGTGCTTGTT
CTTGCCGCGGATGGTGCGCTGGGGCAGGGTCAGGATGCGGTTGCTGGCCA
GGCTGCTGCCGGGCTGCTCGATCACGTTCTGCTCGTCCAGGATCTCGCTG
CCGCTGCTGGTGGGCTGCACCTCGTGCACCTCGTCGATGAAGGCCTCCTC
GGTGTCGCTCTGCACGTCGTCCTCGCTCACGTGGTCGCTCACCTCGCTGT
CGCTGTCCTCGCCCACCAGCTCGTCGTCGCTCTGCAGCAGGGCGCTCAGG
ATGTGCTCGTCGTCCAGGCTGCTGCCGTCCAACTTGACCCTCTTGGCAGC
AGGGCCCATGGTGGCAAGCTTGATCTGCGTTCTACGGTGGTCAGACCGAA
GACTGCGACGGTACCGACGCTGGTCGCGCCTCTTATACCCACGTAGAACG
CAGCTCAGCCAATAGAATGAGTGCCAATATGGAATTTCCAGGGGAAAACC
GGGGCGGTGTTACGTTTTGGCTGCCCTTTCACTTCCCATTGACGTGTATT
GGCTCGAGAACGGTACTTTCCCATTAATCAGCTATGGGAAAGTACCGTTT
AAAGGTCACGTTGCATTAGTTTCAATAGCCCATTGACGTCAATGGTGGGA
AAGTACATGGCGTTTTAATTTAATGGCTGGAAAAACCCAATGACTCACCC
CTATTGACCTTATGTACGTGCCAATAATGGGAAAAACCCATTGACTCACC
CCCTATTGACCTTTTGTACTGGGCAAAACCCAATGGAAAGTCCCTATTGA
CTCAGTGTACTTGGCTCCAATGGGACTTTCCTGTTGATTCACCCCTATTG
ACCTTATGTACTGGGCAAAACCCATTGGAAAGTCCCTAATGACTCAGTAT
ATTTAATTAA

### Sequence 8 (SEQ ID NO: 8, 545bp), EF1α Promoter

AGGATCTGCGATCGCTCCGGTGCCCGTCAGTGGGCAGAGCGCACATCGCC
CACAGTCCCCGAGAAGTTGGGGGGAGGGGTCGGCAATTGAACGGGTGCCT
AGAGAAGGTGGCGCGGGGTAAACTGGGAAAGTGATGTCGTGTACTGGCTC
CGCCTTTTTCCCGAGGGTGGGGGAGAACCGTATATAAGTGCAGTAGTCGC
CGTGAACGTTCTTTTTCGCAACGGGTTTGCCGCCAGAACACAGCTGAAGC
TTCGAGGGGCTCGCATCTCTCCTTCACGCGCCCGCCGCCCTACCTGAGGC
CGCCATCCACGCCGGTTGAGTCGCGTTCTGCCGCCTCCCGCCTGTGGTGC
CTCCTGAACTGCGTCCGCCGTCTAGGTAAGTTTAAAGCTCAGGTCGAGAC
CGGGCCTTTGTCCGGCGCTCCCTTGGAGCCTACCTAGACTCAGCCGGCTC
TCCACGCTTTGCCTGACCCTGCTTGCTCAACTCTACGTCTTTGTTTCGTT
TTCTGTTCTGCGCCGTTACAGATCCAAGCTGTGACCGGCGCCTAC

### Sequence 9 (SEQ ID NO: 9, 720bp), a Sequence Encoding EGFP

ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCA
TGGACGAGCTGTACAAGTAA

### Sequence 10 (SEQ ID NO: 10, 936bp), a Sequence Encoding Luc Luciferase

ATGACTTCGAAAGTTTATGATCCAGAACAAAGGAAACGGATGATAACTGG
TCCGCAGTGGTGGGCCAGATGTAAACAAATGAATGTTCTTGATTCATTTA
TTAATTATTATGATTCAGAAAAACATGCAGAAAATGCTGTTATTTTTTTA
CATGGTAACGCGGCCTCTTCTTATTTATGGCGACATGTTGTGCCACATAT
TGAGCCAGTAGCGCGGTGTATTATACCAGACCTTATTGGTATGGGCAAAT
CAGGCAAATCTGGTAATGGTTCTTATAGGTTACTTGATCATTACAAATAT
CTTACTGCATGGTTTGAACTTCTTAATTTACCAAAGAAGATCATTTTTGT
CGGCCATGATTGGGGTGCTTGTTTGGCATTTCATTATAGCTATGAGCATC
AAGATAAGATCAAAGCAATAGTTCACGCTGAAAGTGTAGTAGATGTGATT
GAATCATGGGATGAATGGCCTGATATTGAAGAAGATATTGCGTTGATCAA
ATCTGAAGAAGGAGAAAAAATGGTTTTGGAGAATAACTTCTTCGTGGAAA
CCATGTTGCCATCAAAAATCATGAGAAAGTTAGAACCAGAAGAATTTGCA
GCATATCTTGAACCATTCAAAGAGAAAGGTGAAGTTCGTCGTCCAACATT
ATCATGGCCTCGTGAAATCCCGTTAGTAAAAGGTGGTAAACCTGACGTTG
TACAAATTGTTAGGAATTATAATGCTTATCTACGTGCAAGTGATGATTTA
CCAAAAATGTTTATTGAATCGGACCCAGGATTCTTTTCCAATGCTATTGT
TGAAGGTGCCAAGAAGTTTCCTAATACTGAATTTGTCAAAGTAAAAGGTC
TTCATTTTTCGCAAGAAGATGCACCTGATGAAATGGGAAAATATATCAAA
TCGTTCGTTGAGCGAGTTCTCAAAAATGAACAATAA

### Sequence 11 (SEQ ID NO: 11, 435bp), GM-CSF Gene

ATGTGGCTGCAGAGCCTGCTGCTCTTGGGCACTGTGGCCTGCAGCATCTC
TGCACCCGCCCGCTCGCCCAGCCCCAGCACGCAGCCCTGGGAGCATGTGA
ATGCCATCCAGGAGGCCCGGCGTCTCCTGAACCTGAGTAGAGACACTGCT
GCTGAGATGAATGAAACAGTAGAAGTCATCTCAGAAATGTTTGACCTCCA
GGAGCCGACCTGCCTACAGACCCGCCTGGAGCTGTACAAGCAGGGCCTGC
GGGGCAGCCTCACCAAGCTCAAGGGCCCCTTGACCATGATGGCCAGCCAC
TACAAGCAGCACTGCCCTCCAACCCCGGAAACTTCCTGTGCAACCCAGAT
TATCACCTTTGAAAGTTTCAAAGAGAACCTGAAGGACTTTCTGCTTGTCA
TCCCCTTTGACTGCTGGGAGCCAGTCCAGGAGTGA

### Sequence 12 (SEQ ID NO: 12, 20bp), Primer PB-F

GCGACAACATCAGCAACATC

### Sequence 13 (SEQ ID NO: 13, 20bp), Primer PB-R

CTTCTTCATCACGGGCTCCT

### Sequence 14 (SEQ ID NO: 14, 17bp), Primer Actin-F

GTTGTCGACGACGAGCG

### Sequence 15 (SEQ ID NO: 15, 17bp), Primer Actin-R

GCACAGAGCCTCGCCTT

### Sequence 16 (SEQ ID NO: 16, 1542bp), a Sequence Encoding CAR19

ATGGCCTTACCAGTGACCGCCTTGCTCCTGCCGCTGGCCTTGCTGCTCCA
CGCCGCCAGGCCGAGCGACATCCAGATGACACAGACTACATCCTCCCTGT
CTGCCTCTCTGGGAGACAGAGTCACCATCAGTTGCAGGGCAAGTCAGGAC
ATTAGTAAATATTTAAATTGGTATCAGCAGAAACCAGATGGAACTGTTAA
ACTCCTGATCTACCATACATCAAGATTACACTCAGGAGTCCCATCAAGGT
TCAGTGGCAGTGGGTCTGGAACAGATTATTCTCTCACCATTAGCAACCTG
GAGCAAGAAGATATTGCCACTTACTTTTGCCAACAGGGTAATACGCTTCC
GTACACGTTCGGAGGGGGGACTAAGTTGGAAATAACAGGCTCCACCTCTG
GATCCGGCAAGCCCGGATCTGGCGAGGGATCCACCAAGGGCGAGGTGAAA
CTGCAGGAGTCAGGACCTGGCCTGGTGGCGCCCTCACAGAGCCTGTCCGT
CACATGCACTGTCTCAGGGGTCTCATTACCCGACTATGGTGTAAGCTGGA
TTCGCCAGCCTCCACGAAAGGGTCTGGAGTGGCTGGGAGTAATATGGGGT
AGTGAAACCACATACTATAATTCAGCTCTCAAATCCAGACTGACCATCAT
CAAGGACAACTCCAAGAGCCAAGTTTTCTTAAAAATGAACAGTCTGCAAA
CTGATGACACAGCCATTTACTACTGTGCCAAACATTATTACTACGGTGGT
AGCTATGCTATGGACTACTGGGGTCAAGGAACCTCAGTCACCGTCTCCTC
AGCGGCCGCATTCGTGCCGGTCTTCCTGCCAGCGAAGCCCACCACGACGC
CAGCGCCGCGACCACCAACACCGGCGCCCACCATCGCGTCGCAGCCCCTG
TCCCTGCGCCCAGAGGCGTGCCGGCCAGCGGCGGGGGGCGCAGTGCACAC
GAGGGGGCTGGACTTCGCCTGTGATATCTACATCTGGGCGCCCCTGGCCG
GGACTTGTGGGGTCCTTCTCCTGTCACTGGTTATCACCCTTTACTGCAAC
CACAGGAACCGTTTCTCTGTTGTTAAACGGGGCAGAAAGAAGCTCCTGTA
TATATTCAAACAACCATTTATGAGACCAGTACAAACTACTCAAGAGGAAG
ATGGCTGTAGCTGCCGATTTCCAGAAGAAGAAGAAGGAGGATGTGAACTG
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGCTGATAA

### Sequence 17 (SEQ ID NO: 17, 604aa), Amino Acid Sequence of PiggyBac Transposase

MGPAAKRVKLDGSSLDDEHILSALLQSDDELVGEDSDSEVSDHVSEDDVQ
SDTEEAFIDEVHEVQPTSSGSEILDEQNVIEQPGSSLASNRILTLPQRTI
RGKNKHCWSTSKPTRRSRVSALNIVRSQRGPTRMCRNIYDPLLCFKLFFT
DEIISEIVKWTNAEISLKRRESMTSATFRDTNEDEIYAFFGILVMTAVRK
DNHMSTDDLFDRSLSMVYVSVMSRDRFDFLIRCLRMDDKSIRPTLRENDV
FTPVRKIWDLFIHQCIQNYTPGAHLTIDEQLLGFRGRCPFRVYIPNKPSK
YGIKILMMCDSGTKYMINGMPYLGRGTQTNGVPLGEYYVKELSKPVHGSC
RNITCDNWFTSIPLAKNLLQEPYKLTIVGTVRSNKREIPEVLKNSRSRPV
GTSMFCFDGPLTLVSYKPKPAKMVYLLSSCDEDASINESTGKPQMVMYYN
QTKGGVDTLDQMCSVMTCSRKTNRWPMALLYGMINIACINSFIIYSHNVS
SKGEKVQSRKKFMRNLYMGLTSSFMRKRLEAPTLKRYLRDNISNILPKEV
PGTSDDSTEEPVMKKRTYCTYCPSKIRRKASASCKKCKKVICREHNIDMC
QSCF

### Sequence 18 (SEQ ID NO: 18, 27bp), a Sequence Encoding C-Myc Nuclear Localization Signal

CCTGCTGCCAAGAGGGTCAAGTTGGAC

### SPECIFIC MODES FOR CARRYING OUT THE INVENTION

The embodiments of the invention are illustrated by reference to the following examples. A person skilled in the art would understand that the following examples are used only for illustrating the invention, but not intended to limit the protection scope of the present invention. In the case where the concrete techniques or conditions are not indicated in the examples, the examples are carried out according to the techniques or conditions described in the documents in the art (see, for example, Sambrook J et al., Molecular Cloning: A Laboratory Manual (Third. Edition), translated by Huang Peitang et al., Science Press) or according to product manuals. The reagents or devices, the manufacturers of which are not indicated, are the conventional products that are commercially available.

### EXAMPLE 1

### Construction of pNB Vector

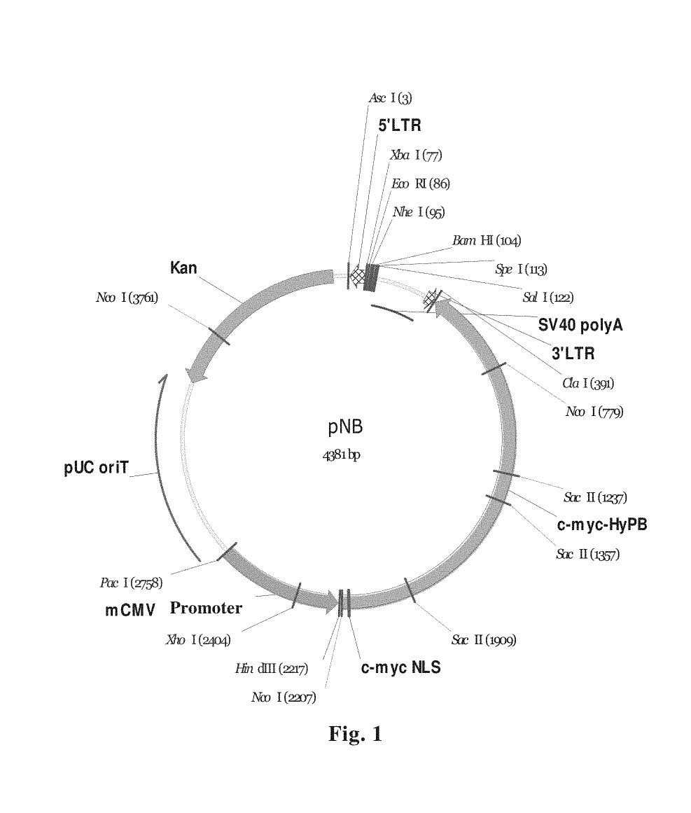

A 5′-terminal repeat sequence of a PiggyBac transposon (SEQ ID NO: 1), a multiple cloning site (SEQ ID NO: 2), a polyA tailing signal sequence (SEQ ID NO: 3), a 3′-terminal repeat sequence of a PiggyBac transposon (SEQ ID NO: 4), sequence encoding PiggyBac transposase and comprising a sequence encoding a c-myc nuclear localization signal (SEQ ID NO: 5), and a CMV promoter sequence (SEQ ID NO: 6) were connected in order thereby to form a long sequence (SEQ ID NO: 7), wherein the sequence encoding PiggyBac transposase and comprising a sequence encoding a c-myc nuclear localization signal and the CMV promoter sequence refer to the reverse complementary sequences thereof (the expression “reverse complementary” used herein means that since the direction of the exogenous gene expression cassette is opposite to the direction of the PB gene expression cassette, the reverse complementary sequences of the sequence encoding PiggyBac transposase and CMV promoter sequence are shown). The long sequence was synthesized by Shanghai Generay Biotech Co., Ltd, and the restriction sites for AscI and PacI were added to two ends, respectively; and the long sequence was packaged into pUC57 (purchased from Shanghai Generay Biotech Co., Ltd) and designated as pNB vector (see FIG. 1).

### EXAMPLE 2

### Construction of pNB Vector Comprising an Exogenous Gene Expression Cassette

1. A sequence of EF1α promoter was synthesized by Shanghai Generay Biotech Co., Ltd, and the restriction sites for XbaI and EcoRI were added to two ends, respectively; and the sequence was packaged into the pNB vector prepared in Example 1 and designated as pNB328 vector.

The sequence of EF1α promoter is set forth in SEQ ID NO: 8.

2. A sequence encoding EGFP was synthesized by Shanghai Generay Biotech Co., Ltd, and the restriction sites for EcoRI and SalI were added to two ends, respectively; and the sequence was packaged into pNB328 vector and designated as pNB328-EGFP vector.

The sequence encoding EGFP is set forth in SEQ ID NO: 9.

3. A sequence encoding Luc luciferase was synthesized by Shanghai Generay Biotech Co., Ltd, and the restriction sites for EcoRI and SalI were added to two ends, respectively; and the sequence was packaged into pNB328 vector and designated as pNB328-Luc vector.

The sequence encoding Luc luciferase is set forth in SEQ ID NO: 10.

4. A human GM-CSF gene was synthesized by Shanghai Generay Biotech Co., Ltd, and the restriction sites for EcoRI and SalI were added to two ends, respectively; and the sequence was packaged into pNB328 vector and designated as pNB328-GM-CSF vector.

The GM-CSF gene is set forth in SEQ ID NO: 11.

### EXAMPLE 3

### PB Expression-Time Curve Analysis After the Transfection of Jurkat Cells With pNB328 Vector

5×10⁶ low passage Jurkat cells in good growing state (purchased from American type culture collection (ATCC)) were prepared, and by using Lonza 2b-Nucleofector device (which was operated according to the user manual), 6 μg of pNB328 plasmids and 6 μs of PB210PA-1 (which provided the expression plasmid of PB transposase, purchased from System Bioscience Company) plasmids were transfected into nuclei, respectively. The cells were cultured in a 37° C., 5% CO₂ incubator. RNA was extracted 6, 12, 24, 48, and 96 hours, and 15 days after transfection, respectively. The relative expression level of PB transposase was determined by RT-PCR. β-actin was used as internal reference, and the particular primers were as follows:

PB-F: as set forth in SEQ ID NO: 12, PB-R: as set forth in SEQ ID NO: 13;

Actin-F: as set forth in SEQ ID NO: 14, Actin-R: as set forth in SEQ ID NO: 15.

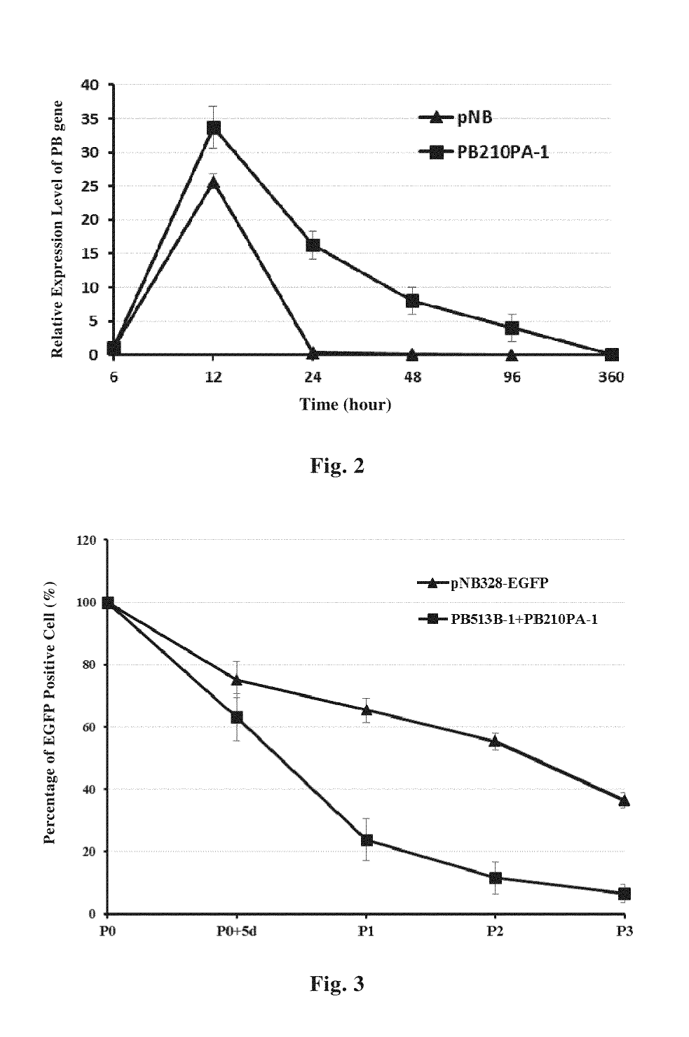

The results show that in the Jurkat cells transfected with pNB328, the amount of mRNA of PB gene reached a peak value at 12 h, and then decreased rapidly, and almost no expression of PB RNA was detected at 24 h after transfection; while in the Jurkat cells transfected with the control plasmid PB210PA-1, the amount of mRNA of PB gene also reached a peak value at 12 h, but decreased slowly, and the expression of PB was still detected at 96 h after transfection (FIG. 2).

The results above show that in the PB self-inactivating mechanism as designed by the inventors (in the PB transposase expression cassette, a polyA tailing signal sequence was located upstream of transposon 3′ ITR), and once PB transposase works, “ITR-exogenous gene expression cassette-ITR” will be cut off from pNB328-EGFP vector and integrated into the host cell genome, and the polyA tailing signal sequence in the PB transposase expression cassette will also be cut off, resulting in incomplete PB transposase expression cassette, and rapid termination of expression.

### EXAMPLE 4

### Quantitative Determination of Integration Efficiency of pNB Vector in Jurkat Cells

5×10⁶ robust Jurkat cells were prepared, and by using Lonza 2b-Nucleofector device, 6 μg of pNB328-EGFP and 5 μg of PB513B-1 (which provides EGFP expression plasmid comprising ITR elements, purchased from System Bioscience Company)+PB210PA-1 plasmid (which provides the expression plasmid of PB transposase) were transfected into nuclei, respectively. The cells were cultured in a 37° C., 5% CO₂ incubator. When the cells reached confluence, they were subjected to passage culture at a ratio of 1:10. A change in percentage of EGFP positive cells was determined by flow cytometry at 12 h (P0), 5 d (P0+5) after transfection, and after 1st passage (P1), 2nd passage (P2), and 3rd passage (P3), respectively.

Since T cells proliferated rapidly, they were passaged at a ratio of 1:10; with cell division, the non-integrating plasmids were lost quickly. Therefore, after 3 passages, the green fluorescent positive cells could be regarded as having stable integration of green fluorescent protein expression cassette. The integration efficiency could be determined by determining the percentage of green fluorescent positive cells using flow cytometry.

As shown in FIG. 3, with the continuous passage at a ratio of 1:10, the percentage of EGFP positive Jurkat cells decreased gradually. After three passages, in the Jurkat T cells transfected with a binary PB transposon system (PB513B-1+PB210PA-1), the percentage of EGFP positive cells was 6.5% (i.e. an integration efficiency of 6.5%); while in the Jurkat T cells transfected with an engineered unary PB transposon system pNB328-EGFP, the percentage of EGFP positive cells was 36.4% (i.e. an integration efficiency of 36.4%).

The above results show that the engineered unary PB transposon system—pNB vector system can efficiently mediate the integration of an exogenous gene.

### EXAMPLE 5

### Analysis on Integration of pNB328-EGFP Vector Into Jurkat Cell and K562 Cell

5×10⁶ low passage Jurkat cells and 5×10⁶ low passage K562 cells (purchased from American type culture collection (ATCC)) both in good growing state were prepared, and by using Lonza 2b-Nucleofector device (which was operated according to the user manual), 6 μg of pNB328-EGFP plasmid and 6 μg of pcDNA3.1-EGFP plasmid (purchased from Addgene Company) were transfected into nuclei, respectively. The cells were cultured in a 37° C., 5% CO₂ incubator. When the cells reached confluence, they were subjected to passage culture at a ratio of 1:10. After three passages, the expression level of green fluorescent protein in the cells was recorded by fluorescence microscopy; 1×10⁵ cells were harvested, and the percentage of EGFP positive cells were determined by flow cytometry.

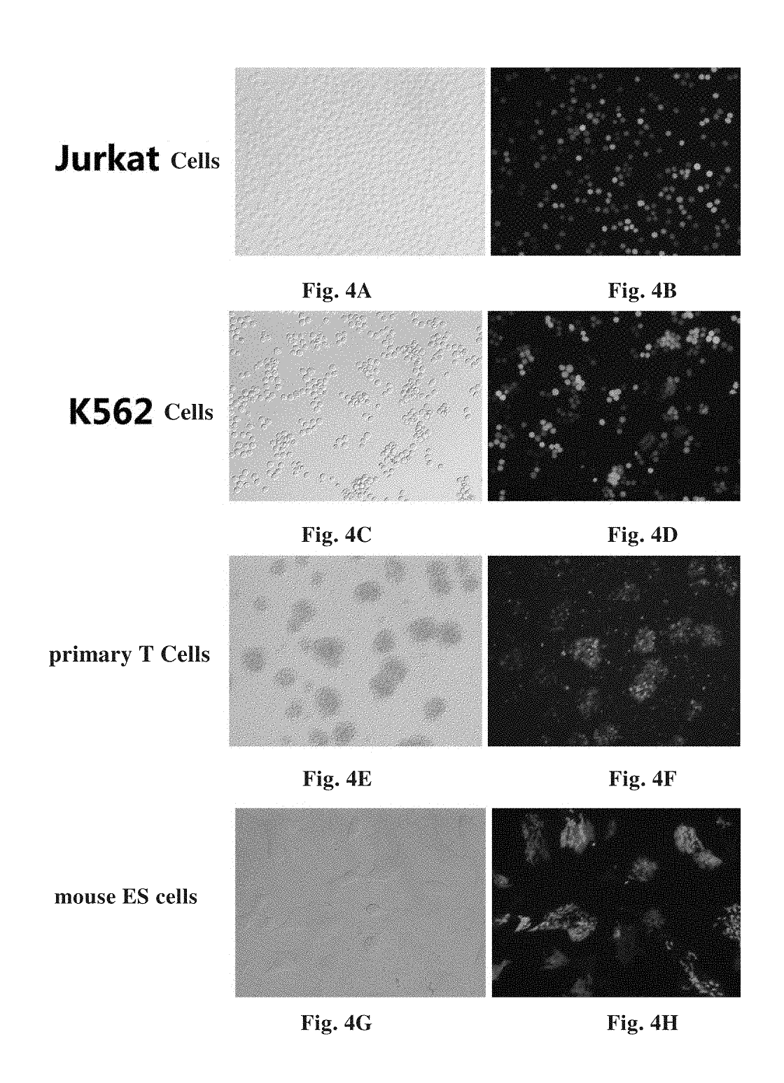

The results show that after three passages of Jurkat cells and K562 cells transfected with the control plasmid pcDNA3.1-EGFP, almost no green fluorescent signal was detected, indicating that the free non-integrating plasmids, which had been transfected into the cells, were completely lost with cell division; in contrast, after three passages of Jurkat cells and K562 cells transfected with pNB328-EGFP, strong green fluorescent signal (FIGS. 4A, 4B, 4C, and 4D) could still be detected, indicating that EGFP expression cassette had been integrated into the cell genome, and could be stably present and expressed with cell division.

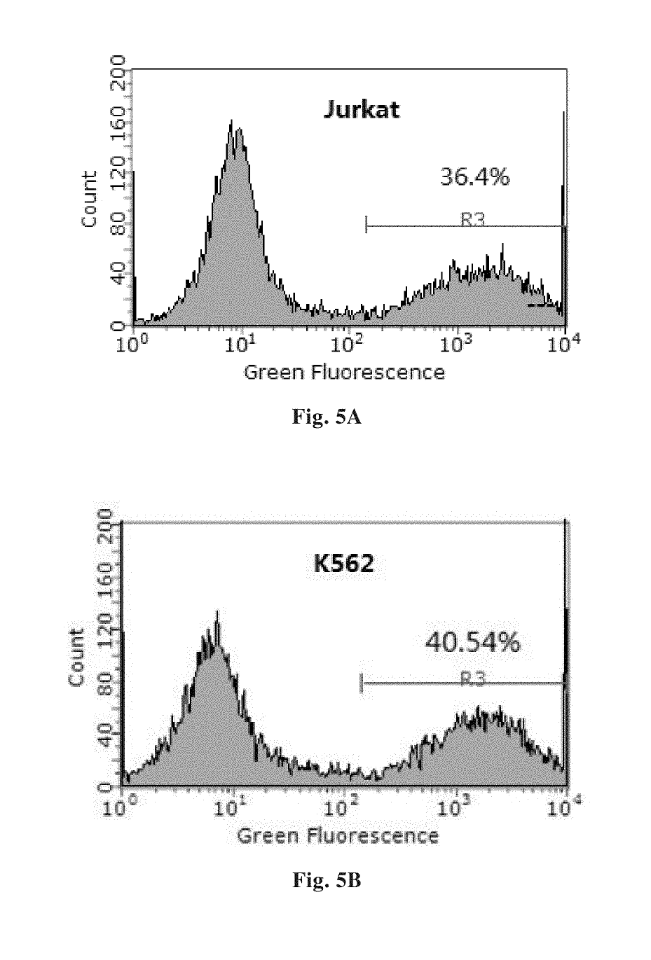

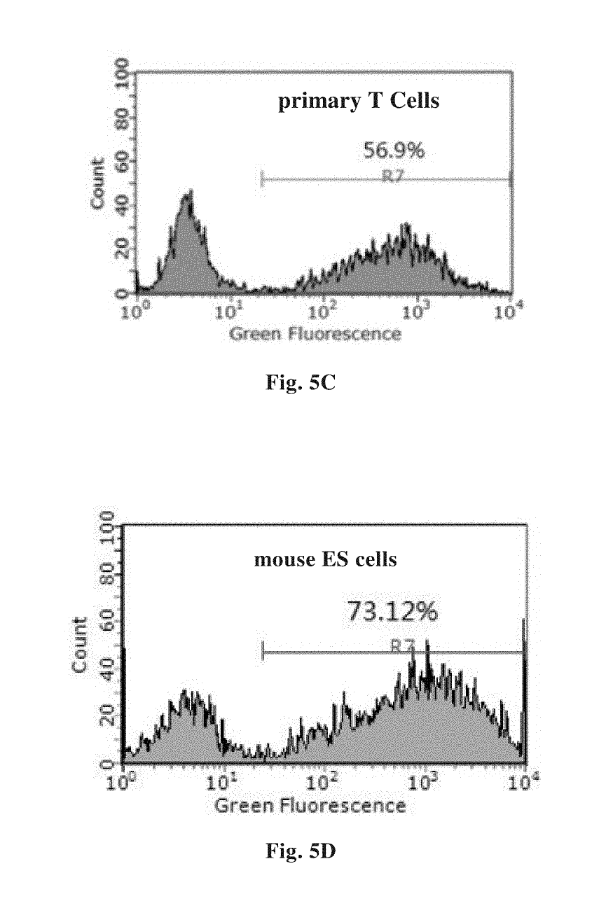

The flow cytometric results show that after transfection of Jurkat cells and K562 cells with pNB328-EGFP plasmid, the integration efficiency was 36.4% and 40.54% (FIGS. 5A, 5B), respectively.

### EXAMPLE 6

### Analysis on Integration of pNB328-EGFP Vector Into Primary T Cells

1×10⁷ freshly isolated peripheral blood mononuclear cells, (PBMC) were prepared, and by using Lonza 2b-Nucleofector device, 6 μg of pNB328-EGFP plasmid and 6 μg of pcDNA3.1-EGFP plasmid were transfected into nuclei, respectively. The cells were cultured in a 37° C., 5% CO₂ incubator. 6 h later, the cells were transferred to a 6-well plate containing 30 ng/mL anti-CD3 antibody and 3000 IU/mL IL-2 (purchased from Novoprotein Company), and cultured in a 37° C., 5% CO₂ incubator. When the cells reached confluence, they were subjected to passage culture at a ratio of 1:10. After three passages, the expression level of green fluorescent protein in the cells was recorded by fluorescence microscopy. In addition, 1×10⁵ cells were harvested, and the percentage of EGFP positive cells were determined by flow cytometry.

The results show that after three passages of primary T cells transfected with the control plasmid pcDNA3.1-EGFP, almost no green fluorescent signal was detected, indicating that the free non-integrating plasmids, which had been transfected into the cells, were completely lost with cell division; in contrast, after three passages of primary T cells transfected with pNB328-EGFP, strong green fluorescent signal could still he detected, indicating that EGFP expression cassette had been integrated into the cell genome, and could be stably present and expressed with cell division (FIGS. 4E and 4F).

The flow cytometric results show that after transfection of primary T cells with pNB328-EGFP plasmid, the integration efficiency was 56.9% (FIG. 5C).

### EXAMPLE 7

### Analysis on Integration of pNB328-EGFP Vector Into Mouse Embryonic Stem Cells

5×10⁶ mouse H9 embryonic stem cells (purchased from ATCC) were prepared, and by using Lonza 2b-Nucleofector device, 6 μg of pNB328-EGFP plasmid was transfected into nuclei. The cells were cultured in a 37° C., 5% CO₂ incubator. When the cells reached confluence, they were subjected to passage culture at a ratio of 1:10. After three passages, the expression level of green fluorescent protein in the cells was recorded by fluorescence microscopy. In addition, 1×10⁵ cells were harvested, and the percentage of EGFP positive cells were determined by flow cytometry.

The results show that after three passages of mouse embryonic stem cells transfected with pNB328-EGFP, strong green fluorescent signal could still be detected, indicating that EGFP expression cassette had been integrated into the cell genome, and could be stably present and expressed with cell division (FIGS. 4G and 4H). The flow cytometric results show that after transfection of mouse embryonic stem cells with pNB328-EGFP plasmid, the integration efficiency was 73.12% (FIG. 5D).

### EXAMPLE 8

### Analysis on Integration of pNB328-Luc Vector Into Tumor Cells

5×10⁶ human hepatocarcinoma. Huh7 cells (purchased from ATCC) were prepared, and by using Lonza 2b-Nucleofector device, 6 μg of pNB328-luc plasmid and 6 μg of pGL4.75-CMV plasmid (purchased from Promega Company) were transfected into nuclei, respectively. The cells were cultured in a 37° C., 5% CO₂ incubator. When the cells reached confluence, they were subjected to passage culture at a ratio of 1:10. After three passages, 1×10⁵ cells were harvested and lysed and the lysed cells were detected by luciferase assay kit (purchased from Promega Company) for activity of Luc luciferase therein.

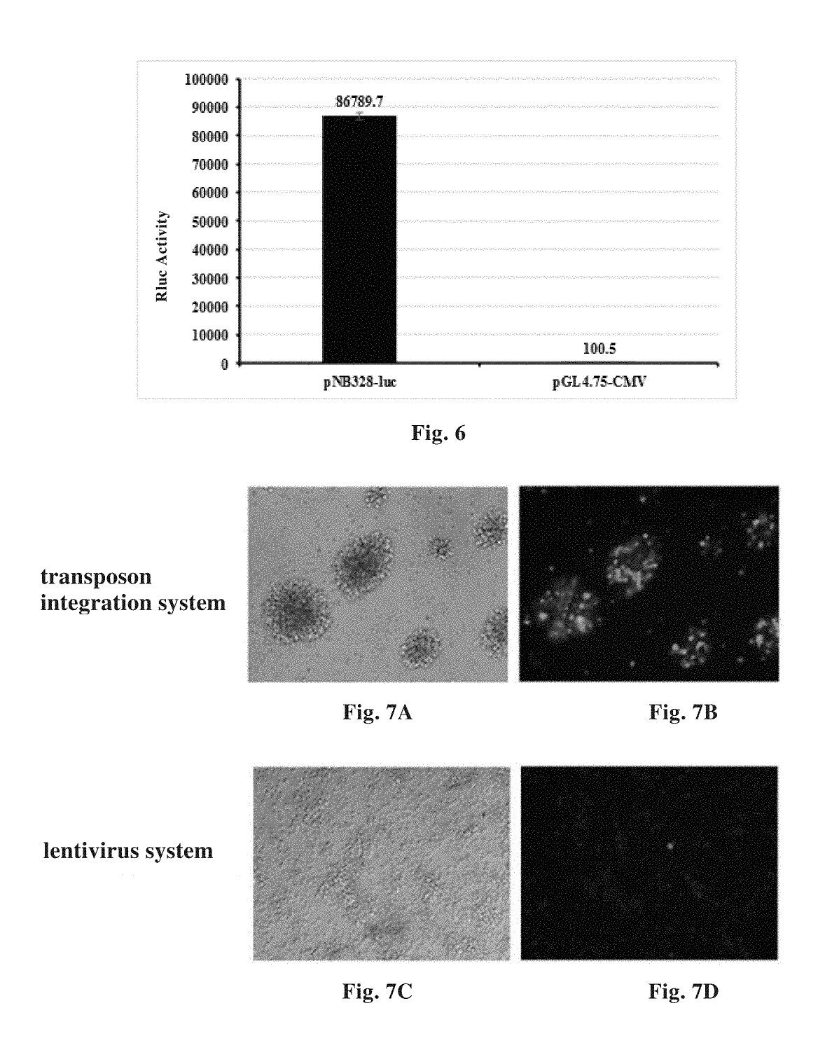

The results show that after three passages of Huh7 cells transfected with the control plasmid pGL4.75-CMV, almost no luciferase activity was detected, indicating that the free non-integrating plasmids, which had been transfected into the cells, were completely lost with cell division; in contrast, after three passages of Huh7 cells transfected with pNB328-luc, strong luciferase activity could still be detected, indicating that luc expression cassette had been integrated into the cell genome, and could be stably present and expressed with cell division (FIG. 6).

### EXAMPLE 9

### Analysis on Integration of pNB328-GM-CSF Vector Into HEK293 Cells

5×10⁶ human HEK293 cells (purchased from ATCC) were prepared, and by using Lonza 2b-Nucleofector device, 6 μg of pNB328-GM-CSF plasmid was transfected into nuclei. The cells were cultured in a 37° C., 5% CO₂ incubator. When the cells reached confluence, they were subjected to passage culture at a ratio of 1:10. After three passages, the supernatant of 1×10⁶ cells cultured for 2 days was harvested, diluted at a certain ratio, and then tested by human GM-CSF ELISA MAX Deluxe assay kit (purchased from Biolegend Company) for the secretion of GM-CSF protein in HEK293 cells transfected with pNB328-GM-CSF plasmid.

The results show that after three passages of HEK293 cells transfected with pNB328-GM-CSF, GM-CSF protein could still be expressed in a high level (1253.7 ng/ml), indicating that GM-CSF expression cassette had been integrated into the cell genome, and could be stably present and expressed with cell division.

### EXAMPLE

### Comparative Analysis on Expression Level of an Exogenous Gene After Integration of pNB328-EGFP Vector Into Primary T Cells

Group 1: 1×10⁷ freshly isolated peripheral blood mononuclear cells (PBMC) were prepared. By using Lonza 2b-Nucleofector device, 6 μg of pNB328-EGFP plasmid and 6 μg of pcDNA3.1-EGFP plasmid were transfected into nuclei, respectively. The cells were cultured in a 37° C., 5% CO₂ incubator. 6 h later, the cells were transferred to a 6-well plate containing 30 ng/mL anti-CD3 antibody and 3000 IU/mL IL-2 (purchased from Novoprotein Company), and cultured in a 37° C., 5% CO₂ incubator.

Group 2: 1×10⁶ PBMC cells from the same healthy person were prepared, and cultured with the stimuli of 30 ng/mL anti-CD3 antibody and 3000 IU/mL IL-2 for 3 days. Then 5×10⁶ activated T cells were infected with a green fluorescent protein-carrying recombinant lentivirus, rLV-EGFP (purchased from Shanghai Telebio Medicine Technology Co., Ltd., MOI=100).

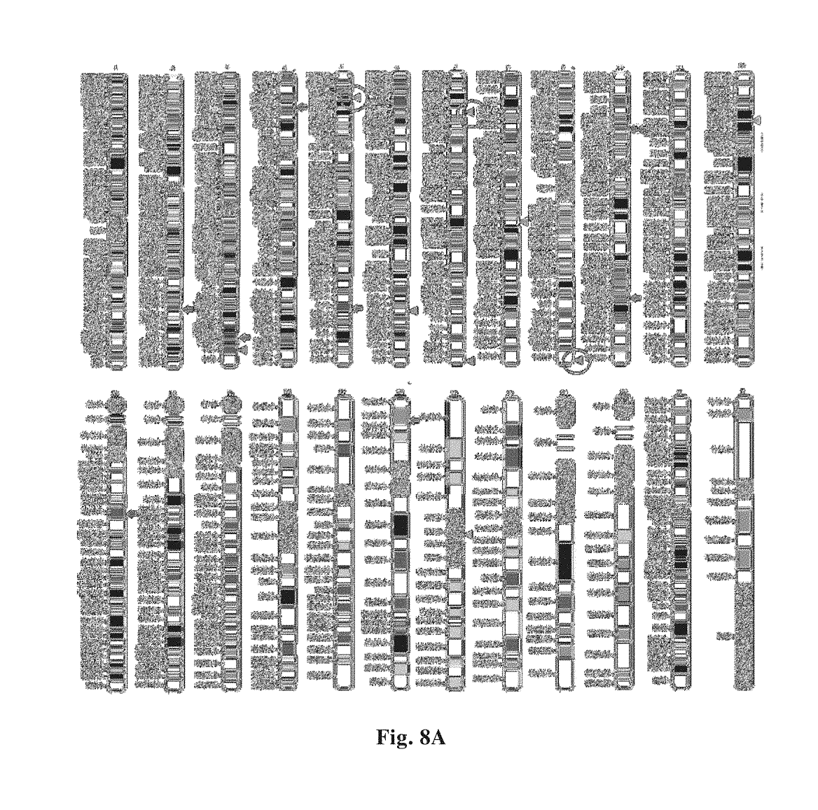

When the cells reached confluence, the cells in Group 1 and 2 were subjected to passage culture at a ratio of 1:10. After three passages, the expression level of green fluorescent protein in the cells was recorded by fluorescence microscopy. In addition, 1×10⁵ cells were collected, and the Mean Fluorescence Intensity (MFI) of EGFP positive cells was determined by flow cytometry. The results show that the T cells integrated with pNB328-EGFP vector, had a high fluorescence intensity (FIGS. 7A and 7B), with a MFI of up to 1507.63; while the T cells infected with the lentivirus, had a low fluorescence intensity, with a MFI of 50.34 (FIGS. 7C and 7D); the MFI of the former was nearly 29 folds higher than that of the later. The results show that after the integration of an exogenous gene into the transfected primary T cells via the pNB328-EGFP vector, it could promote efficient expression of the exogenous gene.

### EXAMPLE 11

### Analysis on Integration Sites of pNB328-EGFP Vector in Primary T Cells

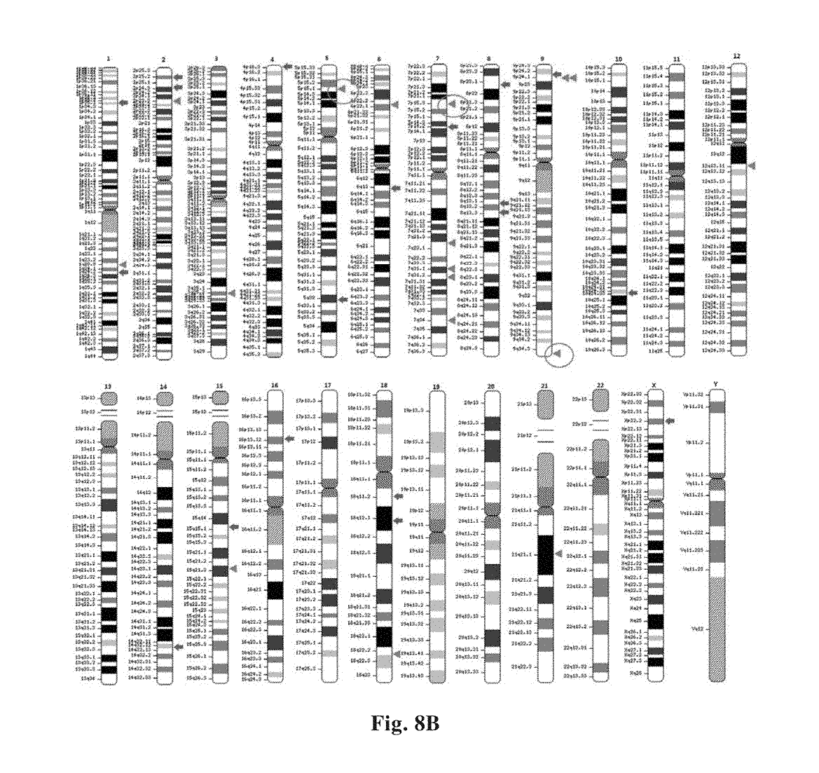

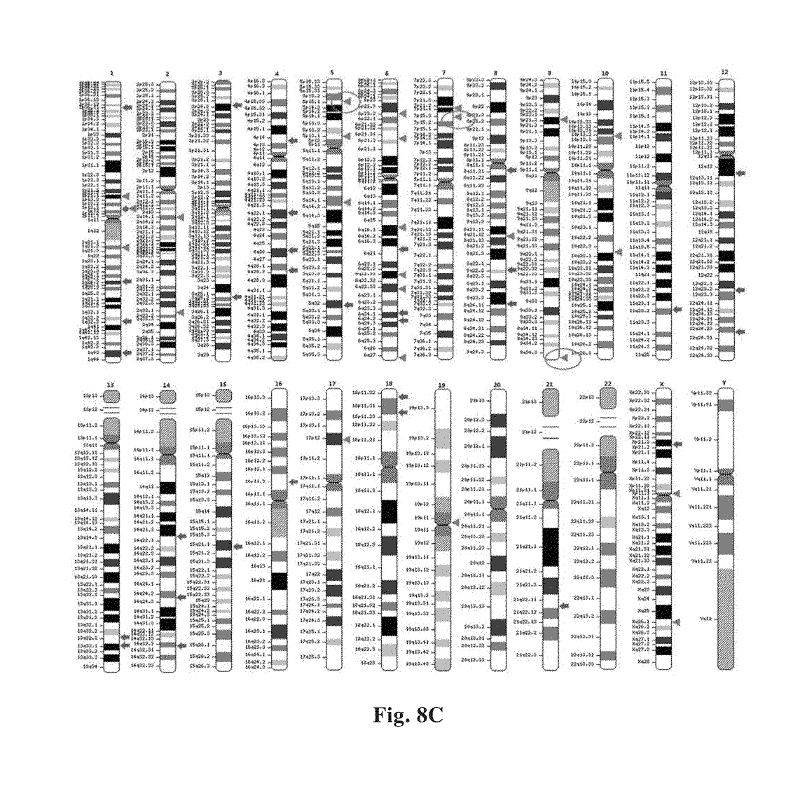

Three fresh PMBCs samples from different persons were prepared, and by using Lonza 2b-Nucleofector device, 6 μg of pNB328-EGFP plasmid was transfected into nuclei. The cells were culture in a 37° C., 5% CO₂ incubator. 6 h later, the cells were transferred to a 6-well plate containing 30 ng/mL anti-CD3 antibody and 3000 IU/mL IL-2 (purchased from Novoprotein Company and cultured in a 37° C., 5% CO₂ incubator. When the cells reached confluence, they were subjected to passage culture at a ratio of 1:10. 5×10⁷ cells were harvested, and genomic DNA was extracted. The whole genome sequencing was performed by Cloud Health Genomics Ltd., and the distribution of the insertion sites of EGFP in the genome was analyzed. The results show that 18 insertion sites were detected for Sample 1, 36 insertion sites were detected for Sample 2, and 61 insertion sites were detected for Sample 3 (an insertion site refers to all the genomic sites that are detected to have genomic integration in a sample; if the integration is detected at the same site repeatedly, the repeated number is defined as detected number) (FIGS. 8A, 8B, and 8C). Surprisingly, the integration hotspots were present in chromosome 5p15.1, chromosome 7p15.1, and chromosome 9q34.3 regions (FIGS. 8A 8B, and 8C, the integration hotspots are circled. Note: in general, the detected number is 1-3 for one site.), the detected number at the adjacent positions of the three regions was 50/66/50 (Note: it refers to the detected number of the integration occurred at chromosome 5p15.1, which is 50 for the first sample, 66 for the second sample, and 50 for the third sample; the following 68/82/64, 78/59/54 can be interpreted in a similar manner), 68/82/64, and 78/59/54 for the three samples, respectively.

Since annotation of the human genome is relatively complete now, whether the region is an intergenic sequence or an intergenic spacer region can be determined by bioinformatics. The results of analysis showed that said three regions all belonged to intergenic spacer regions, and the insertion of an exogenous gene expression cassette therein will not result in inactivation or insertional mutation of relevant genes.

### EXAMPLE 12

### Construction of pNB328-CAR19 and Genetic Modification of Primary T Cell

1. A sequence of chimeric antigen receptor (CAR) against CD19 antigen was synthesized by Shanghai Generay Biotech Co. Ltd., wherein restriction sites for EcoRI and SalI were added to two ends respectively, and the sequence was packaged into pNB328 vector, designated as pNB328-CAR19 vector.

The sequence encoding CAR19 is set forth in SEQ ID NO: 16.

2. 1×10⁷ freshly isolated human PBMCs were prepared, and by using Lonza 2b-Nucleofector device, 6 μg of pNB328-CAR19 plasmid was transfected to nuclei. The cells were cultured in a 37° C., 5% CO₂ incubator; 6 h later, the cells were transferred to a 6-well plate containing 30 ng/mL anti-CD3 antibody and 3000 IU/mL IL-2 (purchased from Novoprotein Company), and cultured in a 37° C., 5% CO₂ incubator. When the cells reached confluence, the T cells genetically modified with CAR19 (CAR19-T) were obtained.

### EXAMPLE 13

### Detection of In Vitro Killing Effect of CAR19-T Cells on Target Cells

CAR19-T and unmodified T cells were co-cultured with Raji cells (purchased from ATCC) at different effector to target cell ratios (8:1, 4:1, 2:1, 1:1, 0.5:1, 0.25:1, 0.125:1, and 0.0625:1), respectively. LDH (lactate dehydrogenase)-Cytotoxicity Assay Kit (Biovision) was used to detect the genetically modified and unmodified T cells for their ability of killing Raji cells in vitro. The method was as follows: the target cells were plated on a 96-well plate (5×10³/well), and the culture medium background control well, volume correction control well, target cell spontaneous LDH release control well, target cell maximum LDH release control well, effector cell spontaneous LDH release control well, and therapeutic group well, were set up in triplicate. The final volume for each well was the same and was not less than 100 μL. After centrifugation at 250 g for 4 min, the cells were incubated at 37° C. in 5% CO₂ for at least 4 h. 10×lysis solution was added to the control well with maximum LDH release, and an equal volume of lysis solution was added to the volume correction control well 45 min prior to centrifugation. After centrifugation again, 50 μL of the supernatant from each well was transferred to a new 96-well plate, where 50 μL of substrate solution per well was added. The plate was incubated at room temperature in dark for 30 min. 50 μL of stop solution was added to each well, and D490 was measured within 1 h. Cytotoxicity (%)=[(D_{experimental well}−D_{culture medium background well})−(D_{effector cell spontaneous LDH release well}−D_{culture medium background well})−(D_{target cell spontaneous LDH release well}−D_{culture medium background well})]/[(D_{target cell maximum LDH release well}−D_{volume correction well})−(D_{target cell spontaneous LDH release well}−D_{culture medium background well})]×¹⁰⁰%

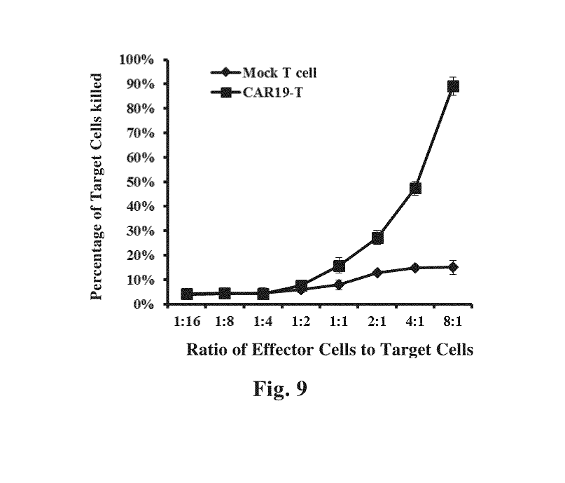

The results show that compared to unmodified. T cells, CAR19-T, obtained by pNB328-CAR19-mediated modification had a significant killing effect on CD19 positive Raji cells (FIG. 9, p<0.001).

A person skilled in the art well knows that Raji cell can be used as a representative of CD19 positive cells. Therefore, CAR19-T, obtained by pNB328-CAR19-mediated modification, could efficiently kill Raji cell, and could also efficiently kill CD19 positive tumor cells, and therefore is of great value in clinical application, for example, it can be used for efficient killing of B cell lymphoma expressing CD19 surface antigen, and has a high therapeutic effect on advanced-stage refractory B cell lymphoma.

Although the embodiments of the invention have been described in detail, a person skilled in the art would understand that a variety of modifications and replacements may be performed to the details according to all the teachings disclosed therein. These changes all fall into the protection scope of the invention. The scope of the invention is defined by the attached claims and any equivalent thereof.

1. A nucleic acid construct, comprising the following elements in order:
a 5′-terminal repeat sequence of a transposon, a polyA tailing signal sequence, a 3′-terminal repeat sequence of a transposon, a sequence encoding a transposase and a promoter controlling expression of the transposase;
wherein the polyA tailing signal sequence has a polyA tailing signal function in both forward and reverse directions; and
wherein the direction of the expression cassette of the transposase is opposite to the direction of an exogenous gene expression cassette.

2. The nucleic acid construct according to claim 1, comprising the following elements in order:
a 5′-terminal repeat sequence of a transposon, a multiple cloning site, a polyA tailing signal sequence, a 3′-terminal repeat sequence of a transposon, a sequence encoding a transposase and a promoter controlling expression of the transposase;
wherein:
the multiple cloning site is used for operably inserting an exogenous gene and optionally a promoter controlling expression of the exogenous gene;
the polyA tailing signal sequence has a polyA tailing signal function in both forward and reverse directions; and
the direction of the expression cassette of the transposase is opposite to the direction of the exogenous gene expression cassette.

3. The nucleic acid construct according to claim 1, wherein the transposon is selected from the group consisting of PiggyBac, sleeping beauty, frog prince, Tn5, Ty, and any combination thereof.

4. The nucleic acid construct according to claim 1, wherein the position of the 5′-terminal repeat sequence of a transposon is interchangeable with the position of the 3′-terminal repeat sequence of a transposon.

5. The nucleic acid construct according to claim 1, wherein the polyA tailing signal sequence is a polyA tailing signal sequence that has a polyA tailing signal function in both forward and reverse directions; or consists of two polyA tailing signal sequences which are connected to each other in an opposite direction and each has a monodirectional polyA tailing signal.

6. The nucleic acid construct according to claim 1, wherein:
the 5′-terminal repeat sequence of a transposon is 5′-terminal repeat sequence of a PiggyBac transposon;
the 3′-terminal repeat sequence of a transposon is 3′-terminal repeat sequence of a PiggyBac transposon; and
the transposase is a PiggyBac transposase.

7. The nucleic acid construct according to claim 6, wherein:
the 5′-terminal repeat sequence of a PiggyBac transposon has a nucleotide sequence set forth in SEQ ID NO: 1; and/or
the 3′-terminal repeat sequence of a PiggyBac transposon has a nucleotide sequence set forth in SEQ ID NO: 4; and/or
the PiggyBac transposase has an amino acid sequence set forth in SEQ ID NO: 17.

8. The nucleic acid construct according to claim 1, wherein the sequence encoding a transposase comprises or is operably linked to a single copy of or multiple copies of a sequence encoding a nuclear localization signal.

9. The nucleic acid construct according to claim 2, characterized by one or more of the following items (1)-(3):
(1) the multiple cloning site has a nucleotide sequence set forth in SEQ ID NO: 2;
(2) the polyA tailing signal sequence has a nucleotide sequence set forth in SEQ ID NO: 3;
(3) the promoter is selected from the group consisting of CMV promoter, EF1α promoter, SV40 promoter, Ubiquitin B promoter, CAG promoter, HSP70 promoter, PGK-1 promoter, β-actin promoter, TK promoter and GRP78 promoter.

10. The nucleic acid construct according to claim 1, wherein the nucleic acid construct is operably linked to one or more identical or different exogenous genes, or operably has one or more identical or different exogenous genes inserted, or wherein the multiple cloning site is replaced by one or more identical or different exogenous genes; and wherein each exogenous gene is independently of single copy or multiple copy.

11. A recombinant vector, comprising the nucleic acid construct according to claim 1.

12. A recombinant host cell, comprising the nucleic acid construct according to claim 1.

13-14. (canceled)

15. A method for integrating an exogenous gene expression cassette into a host cell genome, comprising the step of integrating an exogenous gene expression cassette into a host cell genome by using the nucleic acid construct according to claim 1.

16. The method according to claim 15, wherein the nucleic acid construct is introduced into a host cell by a method selected from the group consisting of virus-mediated transformation, microinjection, particle bombardment, gene gun transformation and electroporation.

17. The nucleic acid construct according to claim 8, wherein the sequence encoding a nuclear localization signal is a sequence encoding a c-myc nuclear localization signal.

18. The nucleic acid construct according to claim 10, wherein the exogenous gene is selected from the group consisting of a gene encoding a fluorescein reporter, a gene encoding a luciferase, a gene encoding a naturally occurring functional protein, RNAi gene, artificial chimeric gene and any combination thereof.

19. The nucleic acid construct according to claim 10, wherein the exogenous gene has a sequence set forth in any one or more of SEQ ID NO: 9-11 and 16.

20. The recombinant vector according claim 11, wherein the recombinant vector is a recombinant cloning vector, a recombinant eukaryotic expression vector or a recombinant virus vector.

21. The recombinant host cell according claim 12, wherein the recombinant host cell is a recombinant mammalian cell.

22. The method according to claim 16, wherein the host cell is a mammalian cell.


SEQUENCE LISTING (18 sequences)

SEQ ID NO: 1 - DNA, 67 bp, Artificial
  5′-terminal repeat sequence of a PiggyBac transposon
ttaaccctagaaagataatcatattgtgacgtacgttaaagataatcatgcgtaaaattgacgcatg

SEQ ID NO: 2 - DNA, 51 bp, Artificial
  multiple cloning site
tctagagtcgaattctgagctagcgatggatcctgcactagtgctgtcgac

SEQ ID NO: 3 - DNA, 222 bp, Artificial
  polyA tailing signal sequence
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 4 - DNA, 40 bp, Artificial
  3′-terminal repeat sequence of a PiggyBac transposon
gcatgcgtcaattttacgcagactatctttctagggttaa

SEQ ID NO: 5 - DNA, 1815 bp, Artificial
  sequence encoding PiggyBac transposase and comprising a sequence encoding a c-myc nuclear localization signal
atgggccctgctgccaagagggtcaagttggacggcagcagcctggacgacgagcacatcctgagcgccctgctgcagagcgacgacgagctggtgggcgaggacagcgacagcgaggtgagcgaccacgtgagcgaggacgacgtgcagagcgacaccgaggaggccttcatcgacgaggtgcacgaggtgcagcccaccagcagcggcagcgagatcctggacgagcagaacgtgatcgagcagcccggcagcagcctggccagcaaccgcatcctgaccctgccccagcgcaccatccgcggcaagaacaagcactgctggagcaccagcaagcccacccgccgcagccgcgtgagcgccctgaacatcgtgcgcagccagcgcggccccacccgcatgtgccgcaacatctacgaccccctgctgtgcttcaagctgttcttcaccgacgagatcatcagcgagatcgtgaagtggaccaacgccgagatcagcctgaagcgccgcgagagcatgaccagcgccaccttccgcgacaccaacgaggacgagatctacgccttcttcggcatcctggtgatgaccgccgtgcgcaaggacaaccacatgagcaccgacgacctgttcgaccgcagcctgagcatggtgtacgtgagcgtgatgagccgcgaccgcttcgacttcctgatccgctgcctgcgcatggacgacaagagcatccgccccaccctgcgcgagaacgacgtgttcacccccgtgcgcaagatctgggacctgttcatccaccagtgcatccagaactacacccccggcgcccacctgaccatcgacgagcagctgctgggcttccgcggccgctgccccttccgcgtgtacatccccaacaagcccagcaagtacggcatcaagatcctgatgatgtgcgacagcggcaccaagtacatgatcaacggcatgccctacctgggccgcggcacccagaccaacggcgtgcccctgggcgagtactacgtgaaggagctgagcaagcccgtgcacggcagctgccgcaacatcacctgcgacaactggttcaccagcatccccctggccaagaacctgctgcaggagccctacaagctgaccatcgtgggcaccgtgcgcagcaacaagcgcgagatccccgaggtgctgaagaacagccgcagccgccccgtgggcaccagcatgttctgcttcgacggccccctgaccctggtgagctacaagcccaagcccgccaagatggtgtacctgctgagcagctgcgacgaggacgccagcatcaacgagagcaccggcaagccccagatggtgatgtactacaaccagaccaagggcggcgtggacaccctggaccagatgtgcagcgtgatgacctgcagccgcaagaccaaccgctggcccatggccctgctgtacggcatgatcaacatcgcctgcatcaacagcttcatcatctacagccacaacgtgagcagcaagggcgagaaggtgcagagccgcaagaagttcatgcgcaacctgtacatgggcctgaccagcagcttcatgcgcaagcgcctggaggcccccaccctgaagcgctacctgcgcgacaacatcagcaacatcctgcccaaggaggtgcccggcaccagcgacgacagcaccgaggagcccgtgatgaagaagcgcacctactgcacctactgccccagcaagatccgccgcaaggccagcgccagctgcaagaagtgcaagaaggtgatctgccgcgagcacaacatcgacatgtgccagagctgcttctaa

SEQ ID NO: 6 - DNA, 531 bp, Artificial
  CMV promoter
atatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccattaaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcactcattctattggctgagctgcgttctacgtgggtataagaggcgcgaccagcgtcggtaccgtcgcagtcttcggtctgaccaccgtagaacgcagatc

SEQ ID NO: 7 - DNA, 2760 bp, Artificial
  a long sequence connected in Example 1
ggcgcgccttaaccctagaaagataatcatattgtgacgtacgttaaagataatcatgcgtaaaattgacgcatgtctagagtcgaattctgagctagcgatggatcctgcactagtgctgtcgaccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcatgcgtcaattttacgcagactatctttctagggttaaatcgatttagaagcagctctggcacatgtcgatgttgtgctcgcggcagatcaccttcttgcacttcttgcagctggcgctggccttgcggcggatcttgctggggcagtaggtgcagtaggtgcgcttcttcatcacgggctcctcggtgctgtcgtcgctggtgccgggcacctccttgggcaggatgttgctgatgttgtcgcgcaggtagcgcttcagggtgggggcctccaggcgcttgcgcatgaagctgctggtcaggcccatgtacaggttgcgcatgaacttcttgcggctctgcaccttctcgcccttgctgctcacgttgtggctgtagatgatgaagctgttgatgcaggcgatgttgatcatgccgtacagcagggccatgggccagcggttggtcttgcggctgcaggtcatcacgctgcacatctggtccagggtgtccacgccgcccttggtctggttgtagtacatcaccatctggggcttgccggtgctctcgttgatgctggcgtcctcgtcgcagctgctcagcaggtacaccatcttggcgggcttgggcttgtagctcaccagggtcagggggccgtcgaagcagaacatgctggtgcccacggggcggctgcggctgttcttcagcacctcggggatctcgcgcttgttgctgcgcacggtgcccacgatggtcagcttgtagggctcctgcagcaggttcttggccagggggatgctggtgaaccagttgtcgcaggtgatgttgcggcagctgccgtgcacgggcttgctcagctccttcacgtagtactcgcccaggggcacgccgttggtctgggtgccgcggcccaggtagggcatgccgttgatcatgtacttggtgccgctgtcgcacatcatcaggatcttgatgccgtacttgctgggcttgttggggatgtacacgcggaaggggcagcggccgcggaagcccagcagctgctcgtcgatggtcaggtgggcgccgggggtgtagttctggatgcactggtggatgaacaggtcccagatcttgcgcacgggggtgaacacgtcgttctcgcgcagggtggggcggatgctcttgtcgtccatgcgcaggcagcggatcaggaagtcgaagcggtcgcggctcatcacgctcacgtacaccatgctcaggctgcggtcgaacaggtcgtcggtgctcatgtggttgtccttgcgcacggcggtcatcaccaggatgccgaagaaggcgtagatctcgtcctcgttggtgtcgcggaaggtggcgctggtcatgctctcgcggcgcttcaggctgatctcggcgttggtccacttcacgatctcgctgatgatctcgtcggtgaagaacagcttgaagcacagcagggggtcgtagatgttgcggcacatgcgggtggggccgcgctggctgcgcacgatgttcagggcgctcacgcggctgcggcgggtgggcttgctggtgctccagcagtgcttgttcttgccgcggatggtgcgctggggcagggtcaggatgcggttgctggccaggctgctgccgggctgctcgatcacgttctgctcgtccaggatctcgctgccgctgctggtgggctgcacctcgtgcacctcgtcgatgaaggcctcctcggtgtcgctctgcacgtcgtcctcgctcacgtggtcgctcacctcgctgtcgctgtcctcgcccaccagctcgtcgtcgctctgcagcagggcgctcaggatgtgctcgtcgtccaggctgctgccgtccaacttgaccctcttggcagcagggcccatggtggcaagcttgatctgcgttctacggtggtcagaccgaagactgcgacggtaccgacgctggtcgcgcctcttatacccacgtagaacgcagctcagccaatagaatgagtgccaatatggaatttccaggggaaaaccggggcggtgttacgttttggctgccctttcacttcccattgacgtgtattggctcgagaacggtactttcccattaatcagctatgggaaagtaccgtttaaaggtcacgttgcattagtttcaatagcccattgacgtcaatggtgggaaagtacatggcgttttaatttaatggctggaaaaacccaatgactcacccctattgaccttatgtacgtgccaataatgggaaaaacccattgactcaccccctattgaccttttgtactgggcaaaacccaatggaaagtccctattgactcagtgtacttggctccaatgggactttcctgttgattcacccctattgaccttatgtactgggcaaaacccattggaaagtccctaatgactcagtatatttaattaa

SEQ ID NO: 8 - DNA, 545 bp, Artificial
  EF1a promoter
aggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 9 - DNA, 720 bp, Artificial
  a sequence encoding EGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 10 - DNA, 936 bp, Artificial
  a sequence encoding Luc luciferase
atgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataa

SEQ ID NO: 11 - DNA, 435 bp, Artificial
  GM-CSF gene
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 12 - DNA, 20 bp, Artificial
  Primer PB-F
gcgacaacatcagcaacatc

SEQ ID NO: 13 - DNA, 20 bp, Artificial
  Primer PB-R
cttcttcatcacgggctcct

SEQ ID NO: 14 - DNA, 17 bp, Artificial
  Primer Actin-F
gttgtcgacgacgagcg

SEQ ID NO: 15 - DNA, 17 bp, Artificial
  Primer Actin-R
gcacagagcctcgcctt

SEQ ID NO: 16 - DNA, 1542 bp, Artificial
  a sequence encoding CAR19
atggccttaccagtgaccgccttgctcctgccgctggccttgctgctccacgccgccaggccgagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcattcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaaccacaggaaccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgataa

SEQ ID NO: 17 - protein, 604 aa, Artificial
  amino acid sequence of a PiggyBac transposase comprising a c-myc nuclear localization signal
MGPAAKRVKLDGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKPTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMGLTSSFMRKRLEAPTLKRYLRDNISNILPKEVPGTSDDSTEEPVMKKRTYCTYCPSKIRRKASASCKKCKKVICREHNIDMCQSCF

SEQ ID NO: 18 - DNA, 27 bp, Artificial
  a sequence encoding c-myc nuclear localization signal
cctgctgccaagagggtcaagttggac